(19) United States
(12) Patent Application Publication
(10) Pub. No.: US 2026/0060556 A1
(43) Pub. Date: Mar. 5, 2026

(54) NON-INVASIVE VITAL SIGN MONITORING METHOD, ELECTRONIC DEVICE AND COMPUTER PROGRAM PRODUCT

(71) Applicant: Wuhan Simshine Intelligent Technology Co., Ltd, Wuhan (CN)

(72) Inventors: Hui Chen, Wuhan (CN); Zhang Xiong, Wuhan (CN); Zhi Zhang, Wuhan (CN); Qingjun Zhang, Wuhan (CN); Guohu Hu, Wuhan (CN)

(73) Assignee: Wuhan Simshine Intelligent Technology Co., Ltd, Wuhan, HB (CN)

(21) Appl. No.: 19/317,117

(22) Filed: Sep. 2, 2025

(30) Foreign Application Priority Data
Sep. 4, 2024 (CN) 202411232902.8

(51) Int. Cl. A61B 5/0205; A61B 5/00; A61B 5/024; A61B 5/08; A61B 5/11; A61B 5/113; G06T 7/00; G06T 7/246; G06T 7/73

(52) U.S. Cl. CPC A61B 5/0205; A61B 5/1114; A61B 5/113; A61B 5/4809; A61B 5/4812; A61B 5/7239; A61B 5/7257; A61B 5/7267; G06T 7/0012; G06T 7/248; G06T 7/73; A61B 5/02433; A61B 5/0816; G06T 2207/10016; G06T 2207/10048; G06T 2207/20056; G06T 2207/20216; G06T 2207/30004; G06T 2207/30168; G06T 2207/30196

(57) ABSTRACT

Provided is a non-invasive vital sign monitoring method, electronic device, and storage medium. The method comprises: acquiring real-time infrared video images in a sleep monitoring environment; performing sleep state monitoring on a subject in the real-time infrared video image to obtain a first target image indicating that the subject has entered a sleep state; performing motion magnification on the first target image according to preset vital sign information to obtain a second target image; performing target region extraction on the second target image to obtain a region associated with the preset vital sign information; and extracting vital signs from the target region to obtain the subject's vital sign information. The invention achieves accurate, non-invasive monitoring of vital signs during sleep.

### TECHNICAL FIELD

The present invention relates to the technical field of vital sign monitoring, and in particular, to a non-invasive vital sign monitoring method, electronic device and computer program product.

### BACKGROUND

In a sleep scenario, real-time monitoring of vital signs is of great significance. Vital signs typically include key indicators such as respiratory rate and heart rate. These signs not only reflect an individual's sleep quality, but also provide a reference for early warning of abnormal conditions and for the assessment of health status. For example, through continuous monitoring of respiratory rate and heart rate, potential health risks such as sleep apnea and arrhythmia can be identified, thereby providing a basis for subsequent intervention and treatment. Therefore, how to accurately acquire vital sign information without affecting the subject's normal sleep has become an important research direction in sleep health management.

In the prior art, common vital sign monitoring methods are mostly contact-based. For example, heart rate signals may be collected by wearable devices such as wristbands or chest patches, or respiratory signals may be collected by embedding pressure sensors in a mattress or pillow. However, such contact-based monitoring methods have certain limitations. On the one hand, wearable devices are in direct contact with the human body, which may cause discomfort in wearing or affect the natural sleep state of the monitored subject. On the other hand, sensors embedded in a mattress or pillow need to maintain close contact with the human body to acquire relatively accurate signals, but signal loss or errors are likely to occur when the subject turns over or changes sleeping posture, resulting in insufficient reliability. Therefore, existing contact-based vital sign monitoring technologies face the technical problem that accuracy and comfort are difficult to achieve simultaneously in sleep monitoring scenario.

The existing Chinese patent CN116636832A discloses a respiration rate monitoring method based on video signals. The method comprises: acquiring a human respiration video; performing preprocessing on the respiration video and then conducting face recognition, and preliminarily extracting a chest region according to the head-to-body ratio of the human body; modeling the extracted chest region to further accurately extract a respiration region; performing filtering on the extracted respiration region and extracting respiratory motion direction features of pixels in the respiration region; performing multi-scale spatial decomposition on the respiration region image according to the respiratory motion direction features to extract respiration phase information; and performing filtering on the extracted respiration phase information and conducting peak detection to estimate the respiration rate. The above patent solution mainly focuses on extracting respiration rate, and fails to simultaneously obtain other key vital signs such as heart rate, resulting in insufficient comprehensiveness of monitoring. In addition, this method relies on recognition and modeling of the chest region, which leads to poor adaptability. In sleep monitoring contexts, it is easily affected by posture changes, bedding occlusion, and lighting conditions, making it difficult to achieve stable and accurate vital sign monitoring. Therefore, its application effect in sleep monitoring contexts is limited.

Accordingly, in sleep monitoring scenario, how to accurately monitor the vital sign information of a subject in a non-invasive manner has become an urgent problem to be solved.

### SUMMARY

In view of the foregoing, the present invention provides a non-invasive vital sign monitoring, electronic device, and computer program product, so as to solve the problem in the prior art that accurate monitoring of the vital sign information of a subject cannot be achieved in a non-invasive manner in sleep monitoring scenario.

In a first aspect, an embodiment of the present disclosure provides a non-invasive vital sign monitoring method, comprising:
acquiring a real-time infrared video image in a sleep monitoring scenario;
performing sleep state monitoring on a monitored subject in the real-time infrared video image to obtain a first target image indicating that the monitored subject has entered a sleep state;
performing motion magnification processing on the first target image according to preset vital sign information to obtain a second target image;
performing target region extraction on the second target image to obtain a target region associated with the preset vital sign information; and,
performing vital sign extraction on the target region to obtain vital sign information of the monitored subject.

In an optional embodiment, the step of performing sleep state monitoring on a monitored subject in the real-time infrared video image to obtain a first target image indicating that the monitored subject has entered a sleep state comprises:
inputting the real-time infrared video image into a pre-trained object detection model to acquire a head position of the monitored subject;
performing object tracking on the head position of the monitored subject to output head tracking box information;
determining, based on the head tracking box information, whether the monitored subject has entered a sleep state; and,
if it is determined that the monitored subject has entered the sleep state, taking the real-time infrared video image in which the monitored subject has entered the sleep state as the first target image.

In an optional embodiment, the step of determining, based on the head tracking box information, whether the monitored subject has entered a sleep state comprises:
acquiring center position coordinates of head tracking boxes based on the head tracking box information;
performing mean value calculation on the center position coordinates to obtain average position information;
determining a head movement state of the monitored subject according to the center position coordinates and the average position information;
acquiring a number of consecutive frames of the real-time infrared video image in which a head of the monitored subject is in a moving state according to the head movement state;
if the number of consecutive frames of the real-time infrared video image in which a head of the monitored subject is in a moving state is greater than a preset frame threshold, identifying that the monitored subject has not entered the sleep state; and,
if the number of consecutive frames of the real-time infrared video image in which the head of the monitored subject is in a moving state is less than or equal to the preset frame threshold, identifying that the monitored subject has entered the sleep state.

In an optional embodiment, the step of performing motion magnification processing on the first target image according to preset vital sign information to obtain a second target image comprises:
performing grayscale conversion on the first target image to acquire a grayscale image;
performing filtering on the grayscale image to output a filtered image;
performing Fourier transform on the filtered image to output an initial frequency-domain image;
enhancing a target frequency in the initial frequency-domain image associated with the preset vital sign information to output an enhanced target frequency-domain image, wherein the preset vital sign information comprises respiratory motion and heart rate motion; and
performing inverse Fourier transform on the target frequency-domain image to output the second target image.

In an optional embodiment, the step of enhancing a target frequency in the initial frequency-domain image associated with the preset vital sign information to output an enhanced target frequency-domain image comprises:
enhancing, according to a first preset frequency range corresponding to respiratory motion, the target frequency in the initial frequency-domain image associated with the respiratory motion to obtain a first frequency-domain image in which the respiratory motion is enhanced;
enhancing, according to a second preset frequency range corresponding to heart rate motion, the target frequency in the initial frequency-domain image associated with the heart rate motion to obtain a second frequency-domain image in which the heart rate motion is enhanced; and,
determining the target frequency-domain image based on the first frequency-domain image and the second frequency-domain image.

In an optional embodiment, the step of performing target region extraction on the second target image to obtain a target region associated with the preset vital sign information comprises:
performing respiratory motion region extraction on the second target image to obtain a respiratory motion region;
performing heart rate motion region extraction on the second target image to obtain a heart rate motion region; and,
determining the target region based on the respiratory motion region and the heart rate motion region.

In an optional embodiment, the step of performing respiratory motion region extraction on the second target image to obtain a respiratory motion region comprises: acquiring a preset number of consecutive frames of the second target image and classifying the second target image into a plurality of image sets;
performing pixel-wise averaging on the image sets to output average images corresponding to each image set;
performing difference calculation on the average images to obtain difference images; and
determining the respiratory motion region based on pixel differences in the difference images and a preset pixel threshold.

In an optional embodiment, the step of determining the respiratory motion region based on pixel differences in the difference image and a preset pixel threshold comprises: taking, according to the pixel differences and the pixel threshold, pixels having pixel differences greater than the pixel threshold as target pixels, and acquiring position information of the target pixels in each difference image;
determining contour regions based on the position information of the target pixels;
comparing areas of the contour regions in the difference images and taking a largest contour region as a target contour region; and,
performing minimum bounding rectangle extraction on the target contour region to obtain the respiratory motion region.

In an optional embodiment, the step of performing heart rate motion region extraction on the second target image to obtain the heart rate motion region comprises:
acquiring center position coordinates and size information of head tracking boxes based on the head tracking box information;
calculating, according to the center position coordinates and the size information, to obtain neck region position information and temple region position information;
performing quality evaluation on neck candidate regions and temple candidate regions according to the neck region position information and the temple region position information to obtain a quality evaluation result; and,
screening, based on the quality evaluation result, the neck candidate region or the temple candidate region as the heart rate motion region.

In an optional embodiment, the step of calculating, according to the center position coordinates and the size information, to obtain neck region position information and temple region position information comprises:
acquiring a width and a height of the head tracking box according to the size information;
determining a first longitudinal boundary of the neck region based on the center position coordinate and the height in combination with a first preset ratio coefficient and a second preset ratio coefficient;
determining a first transverse boundary of the neck region based on the center position coordinate and the width in combination with a third preset ratio coefficient; and
determining the neck region position information according to the first transverse boundary and the first longitudinal boundary.

In an optional embodiment, the step of calculating, according to the center position coordinates and the size information, to obtain neck region position information and temple region position information comprises:
determining, based on the center position coordinate and the height in combination with a fourth preset ratio coefficient, a second transverse boundary and a second longitudinal boundary of the temple region at an outer side position of an upper half portion of the head tracking box; and
determining the temple region position information according to the second transverse boundary and the second longitudinal boundary.

In an optional embodiment, the step of performing quality evaluation on neck candidate regions and temple candidate regions according to the neck region position information and the temple region position information to obtain a quality evaluation result comprises:
extracting pixel data of consecutive frames from the neck candidate regions and the temple candidate regions according to the neck region position information and the temple region position information, and constructing region time-series data for evaluation;
performing imaging quality evaluation on the region time-series data to obtain a first quality metric;
performing energy analysis on the region time-series data according to a preset heart rate-related frequency band to obtain a second quality metric, the energy analysis being configured to characterize the strength relationship between a dominant frequency component and in-band background components;
performing stability evaluation on the region time-series data using a preset time segmentation manner to obtain a third quality metric, the stability evaluation being configured to characterize the consistency of heart rate candidate frequencies across different time segments;
performing artifact detection on the region time-series data to obtain a fourth quality metric, the artifact detection comprising correlation detection with a respiratory rate and harmonics thereof and correlation detection with global body motion; and,
performing weighted calculation based on the first quality metric, the second quality metric, the third quality metric, and the fourth quality metric to obtain the quality evaluation result.

In an optional embodiment, the step of performing vital sign extraction on the target region to obtain the vital sign information of the monitored subject comprises:
calculating a respiratory rate of the monitored subject from the respiratory motion region according to a preset maximum likelihood rule;
calculating a heart rate of the monitored subject from the heart rate motion region according to a preset frequency-domain analysis method; and
determining the vital sign information according to the respiratory rate and the heart rate.

In an optional embodiment, the step of calculating a respiratory rate of the monitored subject from the respiratory motion region according to a preset maximum likelihood rule comprises:
counting a number of preset target pixels in the respiratory motion region to output a numerical sequence;
performing fast Fourier transform on the numerical sequence to convert time-domain feature information of the numerical sequence into frequency-domain feature information;
performing likelihood function modeling on the frequency-domain feature information to obtain a likelihood function; and,
performing logarithmic and derivative operations on the likelihood function to obtain the respiratory rate.

In an optional embodiment, the step of calculating a heart rate of the monitored subject from the heart rate motion region according to a preset frequency-domain analysis method comprises:
extracting pixel intensity variations of the heart rate motion region to construct a one-dimensional signal sequence for frequency-domain analysis;
performing preprocessing on the one-dimensional signal sequence to obtain a preprocessed signal sequence, wherein the preprocessing comprises eliminating a direct current component, removing a trend term, and performing windowing;
performing calculation on the preprocessed signal sequence according to a preset segmental averaged power spectrum estimation method to obtain a power spectrum distribution within a preset frequency range;
determining a target frequency corresponding to a power peak in the power spectrum distribution, and correcting the target frequency by quadratic interpolation to obtain a corrected target frequency; and
converting the corrected target frequency into a heart rate value to obtain the heart rate.

In an optional embodiment, after calculating a respiratory rate of the monitored subject from the respiratory motion region according to the preset maximum likelihood rule to obtain the respiratory rate, the method further comprises: determining a real-time sleep stage of the monitored subject according to the respiratory rate of the monitored subject and a preset respiratory rate range associated with human sleep stages, wherein the sleep stages comprise a deep sleep stage and a light sleep stage;
determining, according to time information, whether the monitored subject is in a nap stage or a nighttime sleep stage;
if the monitored subject is in the nap stage and the real-time sleep stage is the deep sleep stage, acquiring a deep sleep duration;
comparing the deep sleep duration with a preset duration threshold to output a comparison result;
if the monitored subject is in the nighttime sleep stage, acquiring the deep sleep duration and a light sleep duration;
determining a sleep cycle ratio according to the deep sleep duration and the light sleep duration; and,
outputting reminder information according to the comparison result and the sleep cycle ratio.

In an optional embodiment, duration threshold is set according to the age and health condition of the monitored subject, the step of outputting reminder information according to the comparison result and the sleep cycle ratio comprises:
if the deep sleep duration is less than the duration threshold and/or the sleep cycle ratio is abnormal, outputting the reminder information.

In an optional embodiment, the step of acquiring a real-time infrared video image in a sleep monitoring scenario comprises:
acquiring real-time infrared video data including the monitored subject in the sleep monitoring scenario; and,
decomposing the real-time infrared video data to obtain a plurality of frames of the real-time infrared video image.

In a second aspect, an embodiment of the present disclosure provides an electronic device, wherein the electronic device comprises:
at least one processor;
at least one memory; and,
computer program instructions stored in the memory, which, when executed by the processor, implement the method comprising:
acquiring a real-time infrared video image in a sleep monitoring scenario;
performing sleep state monitoring on a monitored subject in the real-time infrared video image to obtain a first target image indicating that the monitored subject has entered a sleep state;
performing motion magnification processing on the first target image according to preset vital sign information to obtain a second target image;
performing target region extraction on the second target image to obtain a target region associated with the preset vital sign information; and,
performing vital sign extraction on the target region to obtain vital sign information of the monitored subject.

In a second aspect, an embodiment of the present disclosure provides a computer program product comprising program instruction that are stored on a computer-readable medium and that, when executed by a processor, cause an electronic device to implement the method comprising:
acquiring a real-time infrared video image in a sleep monitoring scenario;
performing sleep state monitoring on a monitored subject in the real-time infrared video image to obtain a first target image indicating that the monitored subject has entered a sleep state;
performing motion magnification processing on the first target image according to preset vital sign information to obtain a second target image;
performing target region extraction on the second target image to obtain a target region associated with the preset vital sign information; and,
performing vital sign extraction on the target region to obtain vital sign information of the monitored subject.

### Beneficial Effects

The present invention provides a non-invasive vital sign monitoring method, electronic device, and and computer program product. The method comprises: acquiring a real-time infrared video image in a sleep monitoring scenario; performing sleep state monitoring on a monitored subject in the real-time infrared video image to obtain a first target image indicating that the monitored subject has entered a sleep state; performing motion magnification processing on the first target image according to preset vital sign information to obtain a second target image; performing target region extraction on the second target image to obtain a target region associated with the preset vital sign information; and performing vital sign extraction on the target region to obtain vital sign information of the monitored subject.

By acquiring real-time infrared video images in a sleep scenario, the invention avoids the problem that traditional visible-light video is easily affected by lighting conditions, thereby ensuring applicability in low-light or no-light environments. Furthermore, by performing sleep state monitoring on the real-time infrared video image, vital sign extraction is performed only after the monitored subject has entered sleep, reducing interference caused by body movement. On this basis, motion magnification processing is performed on the target image according to the preset vital sign information, so that subtle body surface motion signals such as respiration and heartbeat are significantly enhanced, thereby improving the detectability and signal-to-noise ratio of non-invasive monitoring. Subsequently, by performing target region extraction on the enhanced image, only regions related to vital signs are retained, avoiding interference from environmental background and irrelevant body parts in vital sign extraction. Finally, by performing vital sign extraction on the target region, vital sign information including respiratory rate and heart rate can be simultaneously obtained.

Accordingly, the present invention achieves accurate non-invasive monitoring of vital signs in a sleep scenario.

### BRIEF DESCRIPTION OF THE DRAWINGS

To clarify the technical solutions of the embodiments in the present disclosure, the accompanying drawings used in the embodiments will be briefly introduced. For those skilled in the art, based on these drawings and without exerting inventive effort, other drawings can be obtained. All of these are within the scope of protection of the present disclosure.

FIG. 1 is a schematic flow diagram of the overall operation of the non-invasive vital sign monitoring method according to Embodiment 1 of the present invention;

FIG. 2 is a schematic flow diagram of performing sleep state monitoring on a monitored subject in the real-time infrared video image according to Embodiment 1 of the present invention;

FIG. 3 is a schematic flow diagram of performing motion magnification processing on the first target image according to Embodiment 1 of the present invention;

FIG. 4 is a schematic flow diagram of performing target region extraction on the second target image according to Embodiment 1 of the present invention;

FIG. 5 is a schematic flow diagram of performing respiratory motion region extraction on the second target image according to Embodiment 1 of the present invention;

FIG. 6 is a schematic flow diagram of determining the respiratory motion region according to pixel differences in a difference image and a preset pixel threshold according to Embodiment 1 of the present invention;

FIG. 7 is a schematic flow diagram of performing heart rate motion region extraction on the second target image according to Embodiment 1 of the present invention;

FIG. 8 is a schematic diagram of the relative positional relationship between the heart rate motion region and the head tracking box in Embodiment 1 of the present invention;

FIG. 9 is a schematic diagram of the process for extracting vital signs from the target region;

FIG. 10 is a schematic diagram of the process for calculating the respiratory rate from the respiratory motion region in Embodiment 1 of the present invention;

FIG. 11 is a schematic diagram of the process for calculating the heart rate from the heart rate motion region in Embodiment 1 of the present invention;

FIG. 12 is a schematic diagram of the structure of the electronic device in Embodiment 2 of the present invention;

The labels in the figures are as follows:

1—Head tracking box; 2—Neck region box; 3-1—First temple region box; 3-2—Second temple region box.

### DETAILED DESCRIPTION

In order to make the objectives, technical solutions, and advantages of the embodiments of the present invention clearer, the technical solutions of the embodiments of the present invention will be described clearly and completely below with reference to the accompanying drawings. It should be noted that, in the present application, relational terms such as “first” and “second” are used merely to distinguish one entity or operation from another entity or operation, and do not necessarily require or imply any actual relationship or order between such entities or operations. In the description of the present invention, it should be understood that orientation or positional relationship terms such as “center,” “upper,” “lower,” “front,” “rear,” “left,” “right,” “vertical,” “horizontal,” “top,” “bottom,” “inner,” and “outer” are based on the orientation or positional relationships shown in the drawings, and are merely for convenience of description and simplification of the description, rather than indicating or implying that the device or element referred to must have a particular orientation or be constructed and operated in a particular orientation. Therefore, such terms should not be construed as limiting the present invention. Furthermore, the terms “comprise,” “include,” and any variations thereof are intended to cover non-exclusive inclusion, so that a process, method, article, or device that comprises a list of elements not only comprises those elements, but may also comprise other elements not explicitly listed, or may also comprise inherent elements of such process, method, article, or device. Without further limitation, an element defined by the phrase “comprising . . . ” does not exclude the presence of additional identical elements in a process, method, article, or device that comprises the element. Unless otherwise conflicting, the embodiments of the present invention and the individual features in the embodiments may be combined with each other, and all fall within the protection scope of the present invention.

### Embodiment 1

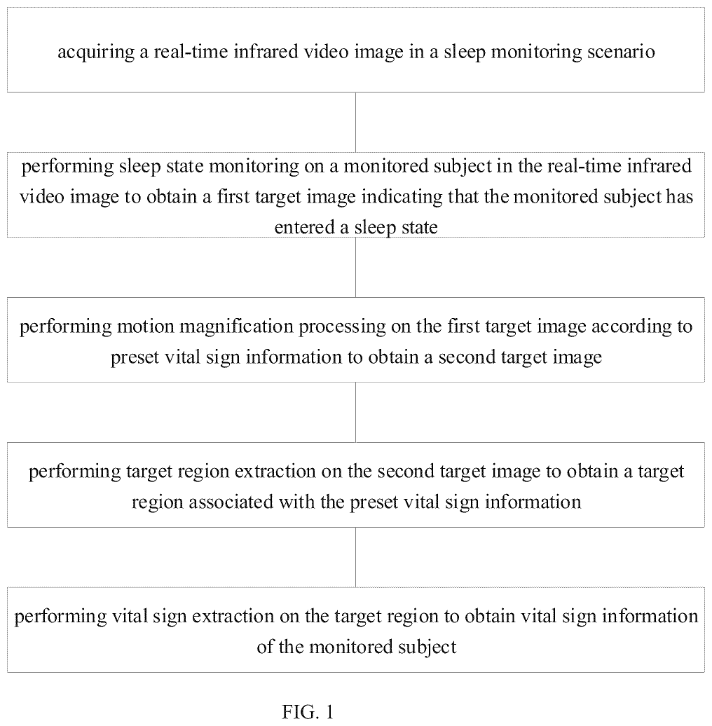

Referring to FIG. 1, Embodiment 1 of the present invention discloses a non-invasive vital sign monitoring method, comprising:
acquiring a real-time infrared video image in a sleep monitoring scenario;

Preferably, the step of acquiring the real-time infrared video image in the sleep scenario comprises:
acquiring real-time infrared video data including the monitored subject in the sleep monitoring scenario; and
decomposing the real-time infrared video data to obtain a plurality of frames of the real-time infrared video image;

Specifically, a high-definition infrared camera with a supplementary light source of a wavelength of 940 nm is used to collect real-time infrared video data including the monitored subject in the sleep scenario, wherein 940 nm is a preferred wavelength, and other wavelengths of supplementary light sources may also be set as needed. The real-time infrared video data is segmented into a plurality of consecutive static images, i.e., real-time infrared video images. The static images may be processed individually or used for further analysis, recognition, or other applications, which helps capture instantaneous situations in the sleep scenario, thereby facilitating a more detailed understanding and processing of the information in the video.
performing sleep state monitoring on a monitored subject in the real-time infrared video image to obtain a first target image indicating that the monitored subject has entered a sleep state;

Specifically, sleep state monitoring is performed on the real-time infrared video image to obtain the first target image indicating that the monitored subject has entered a sleep state, wherein the monitored subject may include infants, adults, and the elderly. The sleep state monitoring comprises: using a pre-trained target monitoring model and a target tracking algorithm to identify a real-time motion state of the head of the monitored subject; if it is identified that the head of the monitored subject remains still continuously, determining that the monitored subject has entered the sleep state and acquiring the first target image corresponding to the sleep state;

Using the head motion state of the monitored subject for sleep state monitoring is non-invasive. Compared with traditional physiological signal monitoring, such as heart rate monitoring or respiration monitoring, analysis of the head motion state is easier to implement without requiring direct contact with the body of the monitored subject. For the monitored subject, monitoring the head motion state is usually more comfortable, as no sensors or devices need to be worn, thereby contributing to a more natural and comfortable sleep environment. Meanwhile, the use of pre-trained target monitoring models and target tracking algorithms simplifies implementation, since such models and algorithms can typically learn and recognize the head motion state of the monitored subject automatically, reducing the need for complex manual feature engineering;
performing motion magnification processing on the first target image according to preset vital sign information to obtain a second target image;

Specifically, the motion magnification processing on the first target image comprises: converting the first target image into a frequency-domain image through fast Fourier transform; enhancing a frequency range associated with the preset vital sign information to output the enhanced second target image.

By performing motion magnification processing, subtle variations of motions related to the preset vital sign information can be observed and analyzed more clearly, thereby providing stronger support for subsequent analysis.
performing vital sign extraction on the target region to obtain vital sign information of the monitored subject;

Specifically, motion magnification or a time-domain band-pass method is first employed to enhance weak periodic displacements or brightness variations in the target region caused by respiration or heartbeat. Subsequently, temporal modeling is performed on the enhanced video frame sequence, and variations such as the grayscale mean of the target region in each frame, principal component components, or the variation of white pixel counts after differential thresholding are used as a one-dimensional time-series signal, thereby reflecting respiratory motion or heartbeat micro-movements of the monitored subject;

To improve the stability of the signal, the time-series signal further undergoes detrending, band-pass filtering, and sampling rate control to ensure coverage of the frequency bandwidth of respiration and heart rate. Finally, by means of a frequency-domain analysis method (such as Welch spectrum estimation or fast Fourier transform combined with peak interpolation), a dominant frequency component is extracted from the time-series signal and converted into vital sign parameters such as respiratory rate or heart rate. In this way, core vital sign information of the monitored subject is obtained, thereby achieving real-time vital sign monitoring in a non-invasive manner.

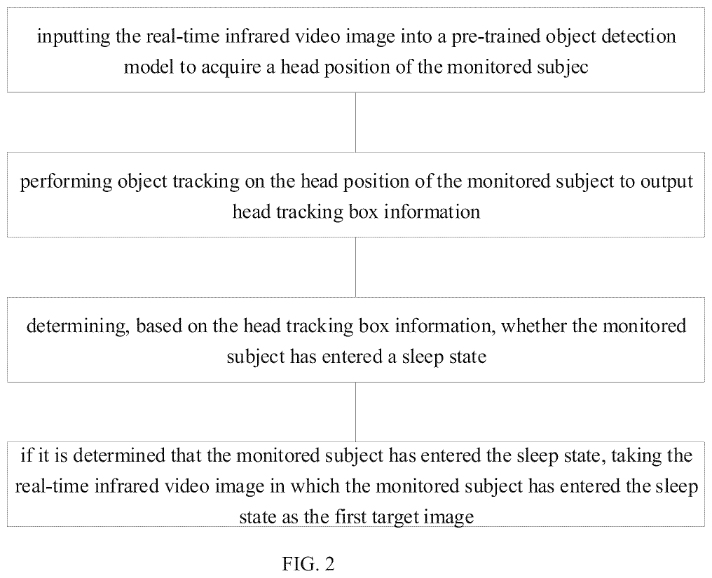

Preferably, referring to FIG. 2, the step of performing sleep state monitoring on a monitored subject in the real-time infrared video image to obtain a first target image indicating that the monitored subject has entered a sleep state comprises:
inputting the real-time infrared video image into a pre-trained object detection model to acquire a head position of the monitored subject;

Specifically, the real-time infrared video image is input into a pre-trained YoloV8s model to obtain the head position of the monitored subject. YoloV8s is an object detection model characterized by high accuracy and real-time performance. First, the real-time infrared video image is passed to the YoloV8s model for processing. The model, having been pre-trained, is capable of recognizing and locating the head of the monitored subject;

The pre-training process is as follows: during training, the model learns features of the monitored subject's head in training images, such as shape and texture, as well as the relationship between the head and the surrounding environment. The model gradually adjusts its parameters to improve its ability to accurately recognize and locate the head of the monitored subject. By comparing with training data labeled with head position information of the monitored subject, the model continuously optimizes its weights so that it can accurately recognize and locate the head of the monitored subject in new images;

By parsing the model output, the bounding box position of the monitored subject's head is obtained for subsequent analysis. The real-time performance of the YoloV8s model makes this method suitable for application scenarios requiring timely response to changes in the sleep state of the monitored subject;
performing object tracking on the head position of the monitored subject to output head tracking box information;

Specifically, an object tracking algorithm is employed to track the head position of the monitored subject, thereby providing real-time and continuous head motion information. In this process, the DeepSort algorithm is adopted for object tracking. DeepSort is a deep learning-based object tracking algorithm that combines object detection and appearance feature embedding, enabling tracking of the target trajectory across video frames;

First, the head position of the monitored subject is passed to the DeepSort algorithm. The DeepSort algorithm uses a Kalman filter for object tracking, predicting the next position of the target and matching it with the actual detection result to achieve continuous tracking of the head of the monitored subject. The output result includes head tracking box information of the monitored subject, i.e., head position, velocity, and other information in each video frame;

By combining object detection with object tracking, this approach can effectively cope with small movements of the monitored subject during sleep, and provide high-precision head position tracking information, thereby supporting subsequent sleep state monitoring;
determining, based on the head tracking box information, whether the monitored subject has entered a sleep state;

Specifically, based on the head tracking box information of the monitored subject, the movement of the head tracking box over a period of time is monitored. By analyzing the velocity and acceleration of the head, it is determined whether the head remains continuously still. In a sleep state, the head of the monitored subject is usually relatively stable, whereas in a wakeful state, the head exhibits small movements. By observing the temporal changes of the head tracking box, when the monitored subject enters a sleep state, the head movement gradually slows down and the variation of the tracking box becomes stable. In contrast, when the monitored subject is awake, the head shows more movements, resulting in more noticeable changes of the tracking box;

If it is determined that the monitored subject has entered the sleep state, taking the real-time infrared video image in which the monitored subject has entered the sleep state as the first target image.

Preferably, the step of determining, based on the head tracking box information, whether the monitored subject has entered a sleep state comprises:
acquiring center position coordinates of the head tracking box based on the head tracking box information;

Specifically, according to the head tracking box information of the monitored subject, tracking boxes of 300 frames of images are acquired each time. For each acquired tracking box Track(i), its center position coordinate Ci(x, y) is extracted;
performing mean value calculation on the center position coordinates to obtain average position information;

Specifically, by calculating the average coordinate Cavg(x, y) of the 300 center coordinates Ci(x, y), an overall average position is obtained;
determining a head movement state of the monitored subject according to the center position coordinates and the average position information;
acquiring a number of consecutive frames of the real-time infrared video image in which a head of the monitored subject is in a moving state according to the head movement state
if the number of consecutive frames of the real-time infrared video image in which the head of the monitored subject is in a moving state is greater than a preset frame threshold, identifying that the monitored subject has not entered the sleep state;
if the number of consecutive frames of the real-time infrared video image in which the head of the monitored subject is in a moving state is less than or equal to a preset frame threshold, it is identified that the monitored subject has entered the sleep state;

Specifically, the Euclidean distance Dis(i) between the center coordinate Ci(x, y) of each tracking box and the average coordinate Cavg(x, y) is calculated. If the Euclidean distance of a certain frame is greater than a preset threshold Thre1, which is set according to specific requirements, that frame is determined to indicate that the head of the monitored subject is in motion. If the number of frames identified as motion frames exceeds 50, it is determined that the monitored subject has not yet entered the sleep state, and another 300 consecutive frames are taken for re-evaluation. If the number of motion frames is less than 50, it is determined that the monitored subject has entered the sleep state;

By determining whether the monitored subject has entered a sleep state based on the average position of the head tracking box center coordinates and the judgment of head movement states, the accuracy of sleep state determination for the monitored subject is improved by combining real-time monitoring of head movements with analysis of overall motion patterns. The setting of the threshold Thre1 and the motion frame number takes into full account variations in actual sleep scenarios of the monitored subject, thereby providing better applicability to different sleep conditions.

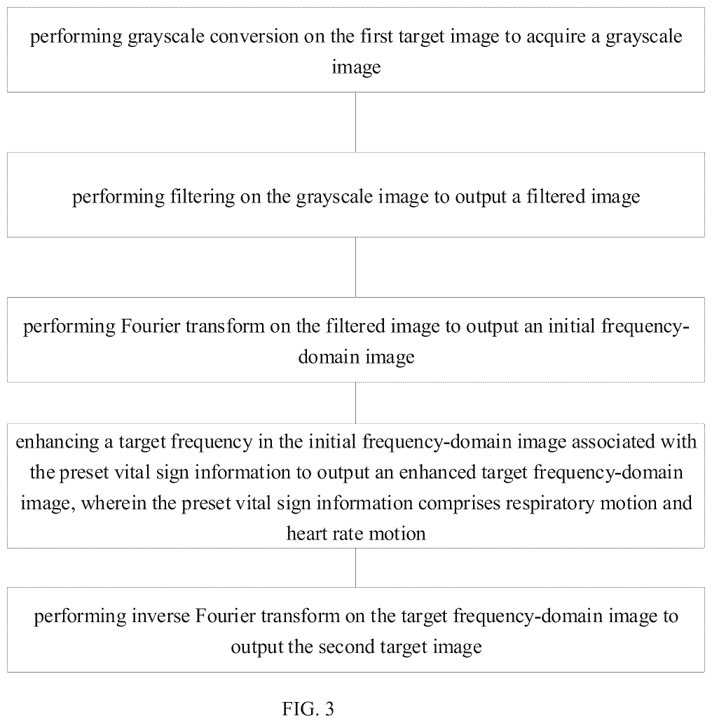

Preferably, referring to FIG. 3, the step of performing motion magnification processing on the first target image according to preset vital sign information to obtain a second target image comprises:
performing grayscale conversion on the first target image to acquire a grayscale image;

Specifically, the first target image is converted into a corresponding grayscale image. The grayscale conversion methods include:

Average method: taking the average value of the red, green, and blue channel values of each pixel as the grayscale value:

$$grayscale value = ( R + G + B ) / 3 ;$$

Weighted average method: considering that different color channels contribute differently to brightness, a weighted average is used:

$$grayscale value = 0.29 * 9 R + 0.587 * G + 0.114 * B ,$$

where the weights are set according to the sensitivity of the human eye to different color channels;

Luminosity method: taking the luminance in the color space as the grayscale value:

$$grayscale value = 0.299 * R + 0.587 * G + 0.114 * B ;$$

All of the above methods are based on the RGB color model, wherein R, G, and B respectively represent the pixel values of the red, green, and blue channels. The choice of method depends on specific requirements and application scenarios, as well as the sensitivity to image brightness information;
performing filtering on the grayscale image to output a filtered image;

Specifically, in order to remove noise in the image, a high-pass filter is applied to each frame of the grayscale image. The main function of the high-pass filter is to emphasize high-frequency information in the image and suppress low-frequency information, thereby highlighting details and edge features. Its principle is to reduce the gradients between pixel values so as to remove low-frequency components in the image while preserving high-frequency components. This is effective in eliminating low-frequency noise introduced by sensor noise or other environmental factors, and helps improve the accuracy of subsequent frequency-domain analysis;
performing Fourier transform on the filtered image to output an initial frequency-domain image;

Specifically, a fast Fourier transform (FFT) is applied to each frame of the grayscale image for frequency-domain analysis. The formula is as follows:

$$F ( u , v ) = \int \int f ( x , y ) {e}^{- 2 π i ( ux + v y )} dxdy$$

where F(u,v) represents the filtered image in the frequency domain, indicating the amplitude and phase information of a specific frequency in the frequency domain; f(x,y) is the grayscale distribution of the original filtered image in the spatial domain, which is a two-dimensional function, with x and y respectively representing the horizontal and vertical coordinates in the image; u and v respectively represent the horizontal and vertical frequencies in the frequency domain; and e⁻²^{^{π}}^{i(ux+vy))} is the complex exponential function describing the phase information in the frequency domain. After the Fourier transform, the initial frequency-domain image is output;
enhancing a target frequency in the initial frequency-domain image associated with the preset vital sign information to output an enhanced target frequency-domain image, wherein the preset vital sign information comprises respiratory motion and heart rate motion;

Specifically, in the initial frequency-domain image, amplitude values within the target frequency range associated with the preset vital sign information are amplified. The preset vital sign information comprises respiratory motion and heart rate motion. To enhance information related to respiratory motion, the amplification is performed by using the following formula:

$$Famplified ( u , v ) = α \times F ( u , v )$$

where Famplified represents the amplified complex representation in the frequency domain, α is an amplification coefficient, and F(u,v) is the complex representation of the original image f(x,y) in the frequency domain. This representation is the result of the Fourier transform and contains the amplitude and phase information of the image at different frequencies. The enhanced target frequency-domain image is then output;
performing inverse Fourier transform on the target frequency-domain image to output the second target image;

Specifically, by applying an inverse Fourier transform, the enhanced target frequency-domain image is converted back into the spatial domain to obtain a new spatial-domain image Fnew(x,y) as the second target image. This process generates a series of new images with noise removed and respiratory motion enhanced, which facilitates clearer observation and analysis of respiration-related motion in the video. Such frequency-domain analysis and processing methods help highlight the frequency range of interest, thereby improving the visibility and analyzability of the respiratory motion of the monitored subject.

Preferably, the step of enhancing a target frequency in the initial frequency-domain image associated with the preset vital sign information to output the enhanced target frequency-domain image comprises:
enhancing, according to a first preset frequency range corresponding to respiratory motion, the target frequency in the initial frequency-domain image associated with the respiratory motion to obtain a first frequency-domain image in which the respiratory motion is enhanced;

Specifically, in order to highlight weak periodic signals corresponding to respiratory motion, detrending and windowing (for example, removing direct current components and low-frequency drift, and then calculating the spectrum in the time domain) are first performed on pixels in the initial frequency-domain image or on one-dimensional time-series signals obtained by prior dimensionality reduction (such as grayscale mean sequences or PCA principal components). In the obtained initial frequency-domain image, a band-pass weighting function or a frequency-domain mask is constructed based on the first preset frequency range (for example, approximately 0.2-1.2 Hz for adults, with appropriate upward adjustment for infants). Frequency components located within this range are amplified, either using a fixed magnification factor or adaptive gain based on frequency energy, while tapering (Gaussian or cosine transition) is applied at the edges of the mask to reduce leakage and ringing artifacts;

To suppress single-pixel noise, small-scale spatial smoothing or median filtering may further be applied to the spectral amplitude map. After the above weighting and amplification processing, a first frequency-domain image in which the respiratory motion is enhanced is obtained. This first frequency-domain image exhibits higher peak energy and more apparent spatial consistency in the respiratory frequency band, thereby facilitating subsequent dominant frequency identification and time-domain reconstruction, and improving the signal-to-noise ratio (SNR) and robustness of respiratory vital sign extraction;
enhancing, according to a second preset frequency range corresponding to heart rate motion, the target frequency in the initial frequency-domain image associated with the heart rate motion to obtain a second frequency-domain image in which the heart rate motion is enhanced;

Specifically, for heart rate micro-movements, which generally have weaker amplitudes and higher frequencies, the sampling rate is first ensured to be sufficient (for example, sampling rate ≥10 Hz, and higher in infant scenarios). Preprocessing such as detrending, filtering, and windowing is performed on the initial frequency-domain image, and the initial frequency-domain representation is calculated. According to the second preset frequency range corresponding to heart rate motion (for example, approximately 0.7-3.0 Hz for adults and 1-4 Hz for infants), a stricter band-pass weighting and gain strategy (with greater gain than respiration) is applied to significantly amplify frequency components associated with heart rate. Meanwhile, a narrow-band adaptive shrinkage is adopted: an energy peak is first identified within the preset frequency band, and then local Gaussian or band-pass weighting centered on the peak position is applied to suppress motion artifacts from adjacent bands. Since heart rate signals are easily affected by head displacement, respiratory harmonics, and environmental noise, spatial weighting (preferentially enhancing anatomical prior regions such as around the carotid artery or temples) is combined with time-frequency smoothing (performing small-bandwidth smoothing in the frequency domain and cross-frame consistency verification). The final output is a second frequency-domain image in which energy in the heart rate frequency band is significantly enhanced and spatial distribution is reasonable, thereby providing a reliable basis for precise identification of the heart rate dominant frequency and subsequent heart rate estimation;
determining the target frequency-domain image based on the first frequency-domain image and the second frequency-domain image;

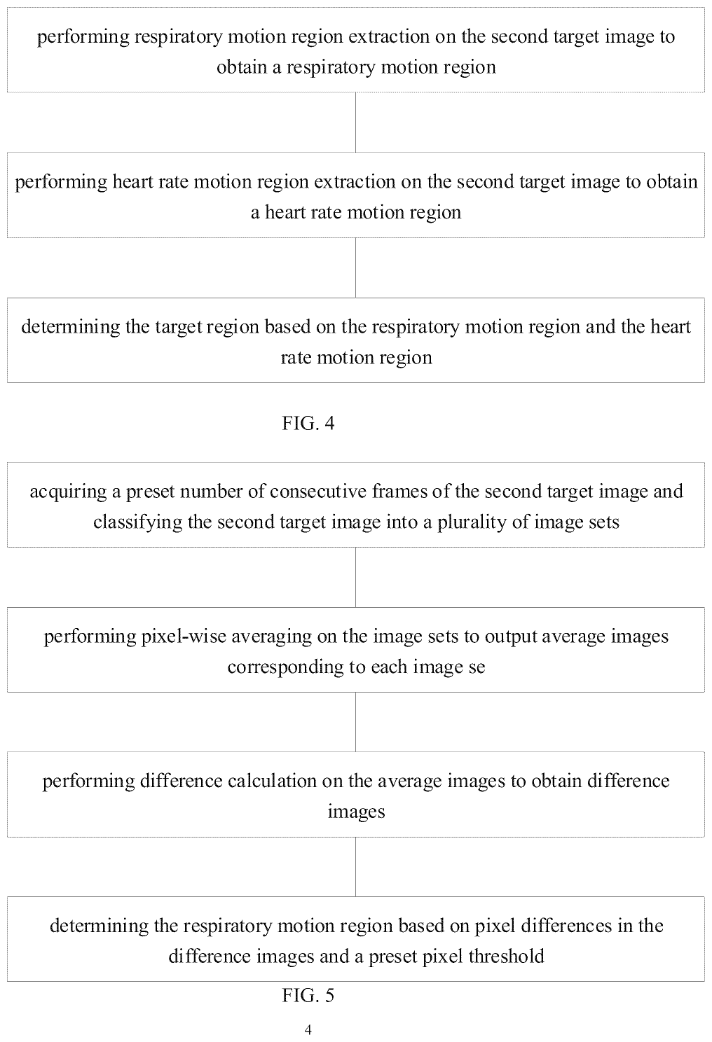

Preferably, referring to FIG. 4, the step of performing target region extraction on the second target image to obtain a target region associated with the preset vital sign information comprises:
performing respiratory motion region extraction on the second target image to obtain a respiratory motion region;

Specifically, after motion magnification is performed on the video frame sequence, a region reflecting respiratory motion is first extracted. The specific approach is to locate pixels or connected regions in which energy is concentrated in the low-frequency band (0.2-1.2 Hz, with appropriate extension for infants) based on the temporal variation characteristics of the enhanced frames. These regions are typically located in the thoracic area. By applying band-pass filtering and differential analysis, a spatial region highly correlated with respiratory motion can be obtained, namely the respiratory motion region. The region extracted in this manner maximally highlights the intensity and stability of respiratory motion, thereby providing reliable input for subsequent vital sign calculation;
performing heart rate motion region extraction on the second target image to obtain a heart rate motion region;

Specifically, in the same enhanced video sequence, analysis is further performed on the high-frequency band (approximately 0.7-4 Hz) to extract regions associated with heart rate. This process is typically carried out through frequency-domain energy peak detection combined with spatial connectivity constraints so as to exclude noise or interference unrelated to heart rate motion. By further screening based on head anatomical priors (such as the temple region and the neck region), a heart rate motion region with stable signals and distinct periodicity can be obtained. This region reflects subtle periodic displacements caused by blood pulsation and serves as a key basis for heart rate vital sign estimation;
determining the target region based on the respiratory motion region and the heart rate motion region.

Preferably, referring to FIG. 5, the step of performing respiratory motion region extraction on the second target image to obtain a respiratory motion region comprises:
acquiring a preset number of consecutive frames of the second target image and classifying the second target image into a plurality of image sets;

Specifically, the second target image refers to consecutive video frames after preprocessing such as stabilization, denoising, grayscaling/normalization, and optional Region of Interest (ROI) cropping. The image sets are clusters of frames obtained by segmenting or grouping these frames in temporal order, either in fixed segments or by overlapping sliding windows;

The purpose of this step is to decompose long-term subtle respiratory motion into a series of “short-term steady-state segments,” thereby providing structured input for subsequent statistical/averaging and variation detection, while suppressing the impact of occasional large movements on later spectral analysis. In practice, consecutive frames are first acquired and subjected to stabilization and illumination normalization. Then, the frame sequence is clustered by fixed-length segments or overlapping sliding windows. The group length and step size may be adaptively set according to the frame rate and the target frequency band (respiration of approximately 0.07-0.40 Hz), such that each group covers at least one effective sample without excessively spanning the respiratory cycle. This grouping enables noise to be more effectively smoothed and feature variations to be more easily localized, thereby simultaneously improving the robustness and efficiency of subsequent processing;
performing pixel-wise averaging on the image sets to output average images corresponding to each image set;

Specifically, pixel-wise averaging refers to performing an arithmetic mean (or robust mean/median) on the pixel values at the same coordinates within the same set, thereby obtaining one average image representing the “short-term steady-state” of the set. The purpose of this step is to reduce sensor noise and random texture disturbances while retaining low-frequency trends caused by slow fluctuations (respiration). In implementation, pixels are aligned within each set before averaging. Depending on the scenario, arithmetic averaging, trimmed mean, or weighted averaging (with weights related to frame clarity or alignment confidence) may be used, in combination with wavelet or Gaussian filtering for further denoising. The obtained average image can be regarded as a “representative frame” in which multiple frames are compressed into one, reducing data volume while improving the signal-to-noise ratio. The beneficial effect is that random noise and micro-jitter are significantly suppressed without losing respiration-related low-frequency information;
performing difference calculation on the average images to obtain difference images;

Specifically, difference image refers to calculating the pixel-wise absolute difference or squared difference between two adjacent average images, which is used to measure the intensity of variation over short periods. The purpose is to highlight the “time-varying components” so that periodic micro-movements caused by thoracoabdominal undulation or surface deformation of coverings are amplified and presented in the images;

In implementation, pixel differences are calculated between adjacent average images, followed by normalization and small-scale morphological filtering (opening/closing operations) to remove isolated noise points. Multi-order differencing or band-pass constraints may further be applied according to the frame rate to attenuate very slow or very fast variations that are unrelated to respiration;

The beneficial effect of the differencing operation is that the background and static textures disappear, and the contrast of the respiration-driven region relative to the whole image is significantly enhanced, thereby providing high-quality input for threshold segmentation and connected component analysis;
determining the respiratory motion region based on pixel differences in the difference images and a preset pixel threshold;

Specifically, the pixel threshold may be a global threshold or an adaptive threshold (such as Otsu or local thresholding). The Region of Interest (ROI) refers to regions with significant differences and with shapes/positions consistent with prior knowledge. This step aggregates pixels with significant variations into connected domains and selects the region most likely corresponding to thoracic undulation as the final monitoring region for subsequent temporal extraction and spectral estimation, i.e., the respiratory motion region;

In implementation, thresholding is applied to the difference images to obtain binary images, followed by connected component labeling. The main connected domain is then selected by combining area, aspect ratio, positional priors (e.g., located within a certain range below the face), and contour stability scores. If necessary, multiple candidate ROIs may be retained and further ranked by SNR or peak prominence for optimal selection;

The beneficial effect is that a stable and interpretable monitoring region consistent with physiological priors is obtained, thereby reducing false detections (such as arm movement or quilt edge jitter) and significantly improving the accuracy and robustness of subsequent vital sign extraction (respiratory rate and phase curve).

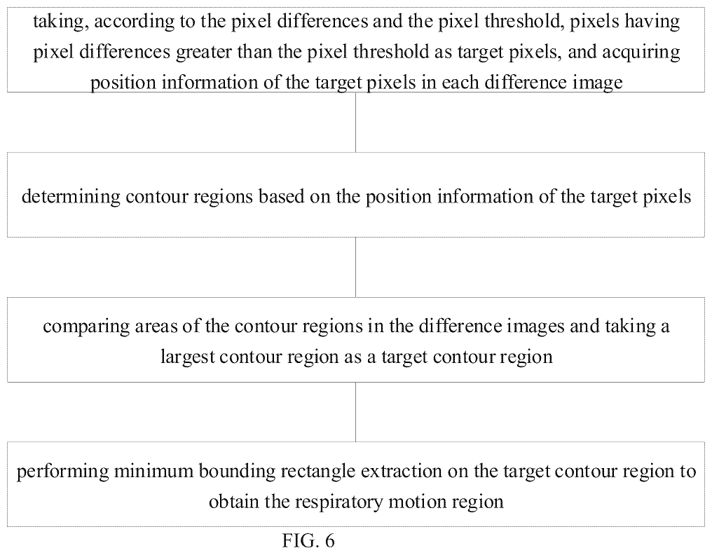

Preferably, referring to FIG. 6, the step of determining the respiratory motion region based on pixel differences in the difference images and a preset pixel threshold comprises:
taking, according to the pixel differences and the pixel threshold, pixels having pixel differences greater than the pixel threshold as target pixels, and acquiring position information of the target pixels in each difference image;

Specifically, the pixel difference refers to the intensity difference of pixels at the same coordinates between adjacent (or fixed-window separated) average images. The pixel threshold may be a fixed value or an adaptive threshold (such as Otsu, percentile thresholding, or local thresholding). When the pixel difference exceeds the threshold, the pixel is identified as a target pixel, indicating that a significant short-term variation (such as brightness/displacement changes caused by thoracoabdominal undulation) exists at that location;

The purpose of this step is to separate “variations” from the background, thereby providing sparse but reliable evidence for subsequent connected-domain or contour construction. In implementation, thresholding is performed on each difference image to generate a binary mask, followed by small-scale morphological opening/closing operations for denoising. All pixels with a value of 1 are then recorded as the target pixel set of that frame with coordinates (x, y), while their time indices are also stored for subsequent temporal consistency analysis;

The beneficial effect of this processing is that noise and gradual illumination changes are significantly suppressed, respiration-related genuine pixel disturbances are preserved, and both the false detection rate and computational load of downstream contour extraction are reduced;
determining contour regions based on the position information of the target pixels;

Specifically, a contour region is a closed boundary or clustered block formed by spatially adjacent target pixels, typically obtained through connected component labeling or edge/contour tracing algorithms. This step aggregates discrete target pixels into spatially coherent candidate motion regions so that they can be filtered using geometric metrics such as area, shape, and position;

In implementation, connected component labeling is first performed on the binary mask to obtain a plurality of candidate blocks. For each block, boundary, centroid, aspect ratio, perimeter, and positional priors (e.g., within a certain range below the face bounding box) are calculated. Hole-filling and erosion-dilation operations are applied to ensure contour closure and boundary smoothing. In this way, scattered noise points are organized into interpretable regional entities, facilitating subsequent stable selection and tracking, and improving spatial consistency and robustness;
comparing areas of the contour regions in the difference images and taking a largest contour region as a target contour region;

Specifically, the contour area refers to either the number of pixels within a candidate region or the polygonal area of the contour. The target contour region refers to the region in a frame or in a batch of difference images that best represents the dominant respiratory motion. Based on the prior knowledge that periodic thoracoabdominal undulation or surface covering movement usually forms the largest and most continuous motion block, small and scattered false targets such as arm jitter or quilt corner swinging are excluded;

In implementation, the area of each candidate contour in every difference image is calculated, and the largest one is selected. To enhance stability, statistical measures across multiple frames (such as mode or median of the maximum areas) may be applied, or a comprehensive scoring function (area*positional weight*shape confidence) may be used for ranking and selection. This ensures the output of a single, stable, and physically reasonable primary motion region, significantly reducing false detections and providing a consistent spatial carrier for subsequent temporal signal construction and spectral estimation;
performing minimum bounding rectangle extraction on the target contour region to obtain the respiratory motion region;

Specifically, the target contour is encapsulated with a regular and compact geometric boundary so that subsequent temporal signal extraction, micro-motion magnification, and quality evaluation within the respiratory motion region can be performed more efficiently and stably. In implementation, the minimum-area bounding rectangle of the target contour is calculated, and a small margin is empirically added to the edges before cropping the respiratory motion region. If necessary, boundary limiting and anti-jitter smoothing (such as Kalman filtering or exponential moving average) are applied to ensure inter-frame continuity;

The beneficial effects are as follows: on the one hand, the true motion is maximally covered while irrelevant background is suppressed, thereby improving the signal-to-noise ratio; on the other hand, the use of a fixed-shape window reduces computational overhead, facilitates long-term tracking, and allows for scoring and selection among multiple respiratory motion regions. Ultimately, a stable and reliable respiratory motion region is obtained as the basis for subsequent vital sign extraction.

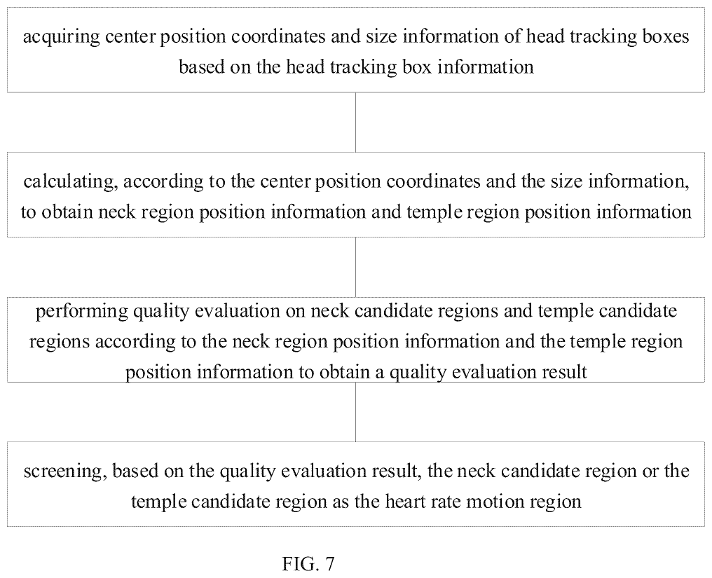

Preferably, referring to FIG. 7, the step of performing heart rate motion region extraction on the second target image to obtain the heart rate motion region comprises:
acquiring center position coordinates and size information of head tracking boxes based on the head tracking box information;

Specifically, the head tracking box information refers to the bounding box of the monitored subject's head in each frame. The center coordinate is the geometric center point of the box, and the size information refers to its width, height, and aspect ratio. The head, as a stable and easily detectable target, is used as a reference origin to provide a coordinate system for inferring heart-rate-sensitive regions such as the neck and temples. This establishes a stable and anti-jitter spatial anchor, thereby reducing drift and false detection in subsequent inference of heart rate motion regions;
calculating, according to the center position coordinates and the size information, to obtain neck region position information and temple region position information;

Specifically, heart-rate-sensitive regions that cannot be directly detected are derived from the head box through geometric rules, thereby narrowing the search space and improving the signal-to-noise ratio. Based on the center position coordinates and the size information, a neck candidate box is inferred below the head box using empirical ratios, while temple candidate boxes are inferred at the upper left and right sides. This enables fast localization of heart-rate-sensitive regions, reduces reliance on lighting and background conditions, and ensures good coverage of candidate regions even in the presence of partial occlusion;
performing quality evaluation on neck candidate regions and temple candidate regions according to the neck region position information and the temple region position information to obtain a quality evaluation result;

Specifically, quality evaluation refers to quantifying the usability of time-series signals in the candidate regions. The heart rate band-pass frequency range is the frequency band where concentrated energy is located (typically around 0.7-4.0 Hz). The goal is to identify the most reliable heart-rate motion signal source among multiple candidate regions, thereby avoiding false peaks caused by partial occlusion, strong illumination, or head movement;

In implementation, a time series is constructed for each candidate region (for example, using RGB-weighted, CHROM, POS chrominance methods, or luminance micro-motion statistics). Band-pass filtering is applied, and Welch or FFT is used to obtain spectral peaks. Evaluation metrics such as SNR, peak prominence, and frequency peak stability are calculated. In addition, ΔHR (heart rate) consistency within sliding windows and flicker suppression scores are combined to form a comprehensive quality score Q. This provides a basis for subsequent automatic selection and continuous output, significantly reducing false detections and abrupt changes;
screening, based on the quality evaluation result, the neck candidate region or the temple candidate region as the heart rate motion region;

Specifically, the heart rate motion-related region is the ROI selected for final heart rate estimation. In time-varying environments, the ROI is adaptively locked to the optimal region to ensure stable and continuous heart rate readings. In implementation, the candidate region with the maximum quality score Q is selected as the current ROI, and hysteresis or sliding averaging is applied to avoid frequent switching. When multiple regions exhibit similar quality or when a single region temporarily fails, strategies such as Q-weighted fusion or fallback mechanisms (e.g., extending the analysis window, enlarging the ROI, or temporarily switching to the contralateral temple/neck) may be triggered. This allows dynamic adaptation to individual and environmental variations, thereby improving the robustness and temporal continuity of heart rate estimation and providing stable input for subsequent HR readings and alarm logic;

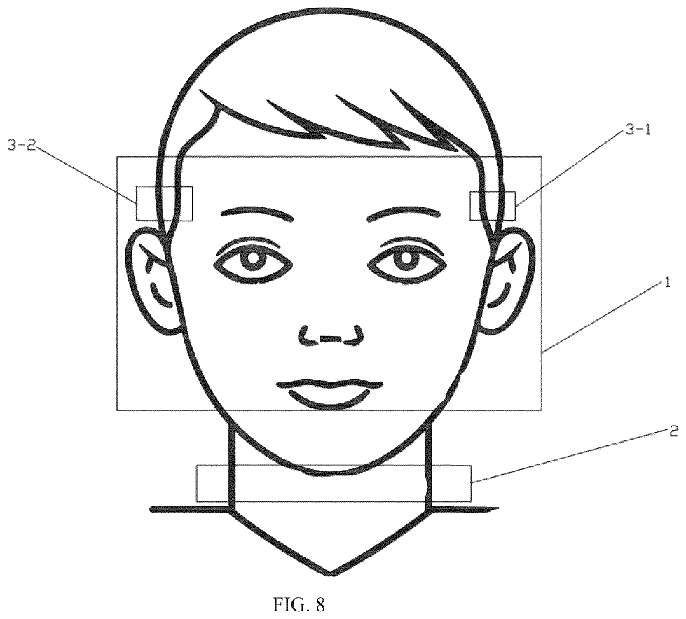

Specifically, referring to FIG. 8, it shows the relative positional relationship between the heart rate motion region and the head tracking box, which includes four boxes: the head tracking box 1, the neck region box 2, the first temple region box 3-1, and the second temple region box 3-2. The head tracking box 1 is the largest rectangular box, covering the entire head region, positioned above the face and surrounding the facial contours. The neck region box 2 is located below the head tracking box, covering the neck area, with a size that is moderate and proportional to the head tracking box. The first temple region box 3-1 is positioned at the upper left corner of the head tracking box, close to the left temple region of the subject. The second temple region box 3-2 is positioned at the upper right corner of the head tracking box, close to the right temple region of the subject;

Preferably, the step of calculating the neck region position information and the temple region position information according to the center position coordinates and the size information comprises:
acquiring a width and a height of the head tracking box according to the size information;

Specifically, the head tracking box is the bounding box of the head output by detection and tracking algorithms. The width and height represent the pixel dimensions of the box, which serve as a scale reference for the head under the current viewpoint. These size parameters are used as a metric reference for all subsequent geometric inferences (such as ROIs for the neck and temples), thereby enabling normalization across different distances and imaging magnifications;

In implementation, the tracker outputs (x_{c}, y_{c}, w, h) for each frame. The values of w and h are subjected to temporal smoothing (e.g., exponential moving average or Kalman filtering), and in cases of occlusion or abrupt changes, the last reliable values are used. This approach reduces the amplification effect of jitter and misdetection on subsequent ratio-based inference, ensuring stable scale estimation that is transferable across subjects and postures;
determining a first longitudinal boundary of the neck region based on the center position coordinate and the height in combination with a first preset ratio coefficient and a second preset ratio coefficient;

Specifically, the first ratio coefficient k₁ and the second ratio coefficient k₂ are dimensionless parameters relative to the head height h, and are used to map the head center point y_{c} to the vertical positions of the upper and lower boundaries of the neck. The purpose is to determine the longitudinal range of the neck, which cannot be directly detected, by utilizing the anatomical prior that the neck is located below the head box and typically occupies a certain proportion of the head height;

In implementation, the boundaries may be defined as yₜₒₚ=y_{c}+k₁*h and y_{bottom}=y_{c}+k₂*h, where the coefficients are obtained from datasets or empirical calibration, with boundary clipping applied to avoid out-of-range errors. When a pitch angle is present, the angle may be estimated from facial landmarks, and small corrections may be applied to k₁ and k₂. In this way, the longitudinal boundaries adaptively scale with distance and head size, thereby reducing ROI displacement caused by individual differences;
determining a first transverse boundary of the neck region based on the center position coordinate and the width in combination with a third preset ratio coefficient;

Specifically, the third ratio coefficient k₃ maps the head width w to the displacement of one lateral boundary (e.g., the left boundary) of the neck region along the horizontal direction. The purpose is to constrain the left-right range of the neck ROI by referencing the lateral scale of the head, so as to cover the area near the carotid arteries while avoiding inclusion of the shoulders or background noise;

In implementation, the boundary may be defined as x_{left}=x_{c}−k₃*w (or, for the right side, x_{right}=x_{c}+k₃*w), such that the transverse boundary scales synchronously with the head width. This ensures appropriate coverage under different camera distances and facial orientations;
determining the neck region position information according to the first transverse boundary and the first longitudinal boundary;

Specifically, the neck region position information is a rectangular or rotated-rectangular ROI (defined by top/bottom/left/right boundaries or by center+width+height+angle) derived from the combination of the first transverse boundary and the first longitudinal boundary. The purpose is to map the geometric priors into a stable ROI that can be directly cropped and analyzed for subsequent signal construction (e.g., rPPG or micro-motion), quality evaluation, and heart rate estimation;

In implementation, the first transverse and longitudinal boundaries are combined into a bounding box, which is expanded by a small margin and subjected to temporal hysteresis or smoothing to prevent inter-frame jitter. When abnormal jumps or a reduction in quality scores are detected, the system may automatically revert to the last stable ROI;

The resulting neck ROI offers three main advantages: scale adaptivity, anatomical consistency, and temporal stability, thereby significantly improving the signal-to-noise ratio and robustness of subsequent vital sign extraction.

Preferably, the step of calculating, according to the center position coordinates and the size information, to obtain neck region position information and temple region position information further comprises:
determining, based on the center position coordinate and the height in combination with a fourth preset ratio coefficient, a second transverse boundary and a second longitudinal boundary of the temple region at an outer side position of an upper half portion of the head tracking box;

Specifically, the fourth ratio coefficient refers to a dimensionless coefficient scaled by the head height h. The “outer side position of the upper half portion of the head tracking box” refers to a narrow band area laterally offset outward from the left and right edges of the upper half of the head, corresponding to the temple location. The purpose is to stably locate the temple region, which cannot be directly detected, by applying computable geometric rules, thereby avoiding interference caused by hair or cheek shadows;

In implementation, starting from the head tracking box (x_{c}, y_{c}, w, h), a vertical strip for the upper half is first defined as yϵ[y_{c}−β₂*h, y_{c}−β₁*h]. Then, the second transverse boundaries on the left and right sides are respectively calculated as x_{R}=x_{c}+w/2+k₄*h and x_{L}=x_{c}−w/2−k₄*h. The second longitudinal boundaries are defined as yₜₒₚ=y_{c}−β₂*h and y_{bottom}=y_{c}−β₁*h, wherein β₁ and β₂ are two preset ratio coefficients relative to head height h;
determining the temple region position information according to the second transverse boundary and the second longitudinal boundary;

Specifically, the second transverse and longitudinal boundaries are converted into a stable ROI that can be directly cropped and subjected to time-series analysis as a candidate region for heart rate extraction. In implementation, the second transverse and longitudinal boundaries are combined to form candidate boxes for the left and right temples. The center and scale of the ROI are smoothed by exponential moving average or Kalman filtering to suppress inter-frame jitter. When the quality of a single side is low, dual-side candidates may be simultaneously generated for subsequent quality evaluation and optimal selection;

The output temple ROI is geometrically consistent, temporally stable, and directly usable for signal construction, thereby reducing the impact of false detections and jitter on heart rate estimation, and providing standardized input for subsequent SNR/peak prominence scoring and automatic switching.

Preferably, the step of performing quality evaluation on neck candidate regions and temple candidate regions according to the neck region position information and the temple region position information to obtain a quality evaluation result comprises:
extracting pixel data of consecutive frames from the neck candidate regions and the temple candidate regions according to the neck region position information and the temple region position information, and constructing region time-series data for evaluation;

Specifically, based on the neck and temple candidate regions, pixels are extracted frame by frame to construct region time-series data. The region time-series data refers to a univariate sequence formed by arranging in temporal order representative statistics of the region in each frame, such as grayscale mean, linear combinations of skin-tone channels, white-pixel counts obtained from differential thresholding, or median magnitudes of optical flow vectors. The purpose is to transform image information into a univariate sequence that reflects subtle pulsatile variations;

In implementation, image stabilization and denoising are first performed, after which the above-mentioned statistics are calculated within the candidate regions. Detrending, normalization, and band-pass pre-filtering are then applied to obtain uniformly sampled and amplitude-comparable sequences. This significantly compresses the data and reduces random noise, providing high-SNR input for subsequent quality evaluation and spectral analysis;
performing imaging quality evaluation on the region time-series data to obtain a first quality metric;

Specifically, imaging quality evaluation on the region time-series data yields the first quality metric. Imaging quality focuses on the question of “whether the signal is clearly observable,” and considers factors such as sharpness, contrast, overexposure or underexposure, proportion of skin pixels, degree of occlusion, and brightness stability. The purpose is to eliminate segments that inherently lack measurability. In implementation, sharpness can be assessed using gradient energy, contrast and exposure can be evaluated with histograms, the effective proportion of skin can be determined using skin-tone ratios or near-infrared reflectance, and occlusion and specular reflection ratios can also be quantified. After normalization, a score on a scale of 0-100 or 0-1 is produced. A higher score indicates better image quality and more stable texture, and therefore a higher likelihood of obtaining reliable heart rate signals in subsequent analysis;
performing energy analysis on the region time-series data according to a preset heart rate-related frequency band to obtain a second quality metric, the energy analysis being configured to characterize the strength relationship between a dominant frequency component and in-band background components;

Specifically, energy analysis within the heart rate-related frequency band yields the second quality metric. The heart rate-related frequency band refers to the range that contains the fundamental pulse frequency (for example, for adults, typically within a few Hertz). The purpose is to evaluate whether the dominant frequency component is sufficiently prominent;

In implementation, the time-series data is first subjected to band-pass filtering, followed by calculation of the power spectrum or autocorrelation sequence. The ratio of the dominant peak energy to the background energy within the band, or the difference in prominence between the primary peak and secondary peaks, is then taken as the score. A larger value of this metric indicates that the heart rate component is more dominant and less affected by interference, and that the region is therefore more suitable for subsequent heart rate estimation;
performing stability evaluation on the region time-series data using a preset time segmentation manner to obtain a third quality metric, the stability evaluation being configured to characterize the consistency of heart rate candidate frequencies across different time segments;

Specifically, stability evaluation based on a preset time segmentation manner yields the third quality metric. Here, stability refers to whether the heart rate candidate frequencies remain consistent across different time segments. The purpose is to avoid misjudgments caused by short-term incidental peaks;

In implementation, the time-series data is divided into multiple segments using a sliding window. For each segment, candidate frequencies and their peak values are extracted. The variance of these frequencies, the offset between adjacent segments, and the median consistency rate are then calculated. Smaller variance and higher overlap rates correspond to higher scores. This metric ensures temporal continuity and reliability of the output, effectively suppressing the influence of transient noise and occasional body movements;
performing artifact detection on the region time-series data to obtain a fourth quality metric, the artifact detection comprising correlation detection with a respiratory rate and harmonics thereof and correlation detection with global body motion;

Specifically, artifact detection on the region time-series data yields the fourth quality metric. Artifacts mainly originate from low-frequency fluctuations caused by respiration and their harmonics, as well as from global body motion. The purpose is to identify “false signals that resemble heart rate.”

In implementation, the respiratory rate is estimated simultaneously, and the correlation between the heart rate candidate frequency and the respiratory frequency and its harmonics is calculated to determine whether the ratios approach simple integer multiples. Global optical flow or frame-wise energy variation is then used to measure body motion intensity, and the correlation with the candidate signal is computed. If the correlation or motion intensity exceeds a preset threshold, the score is reduced. This step significantly reduces false positives where respiratory motion or large-scale body movements might otherwise be mistaken for heart rate;
performing weighted calculation based on the first quality metric, the second quality metric, the third quality metric, and the fourth quality metric to obtain the quality evaluation result;

Specifically, the four metrics are fused through weighted calculation to generate the quality evaluation result. The purpose is to comprehensively integrate imaging quality, dominant frequency prominence, temporal stability, and artifact suppression into a single comparable score;

In implementation, the four metrics are first normalized and then linearly weighted according to empirically determined or offline-calibrated coefficients. Thresholding and hysteresis may be applied where necessary to suppress fluctuations. The output includes a composite quality score and its corresponding level, which can be used to select the optimal region among multiple candidates or to determine whether the current reading is valid. This fusion result adapts to different subjects and illumination conditions, providing robust guidance for subsequent region selection and heart rate estimation.

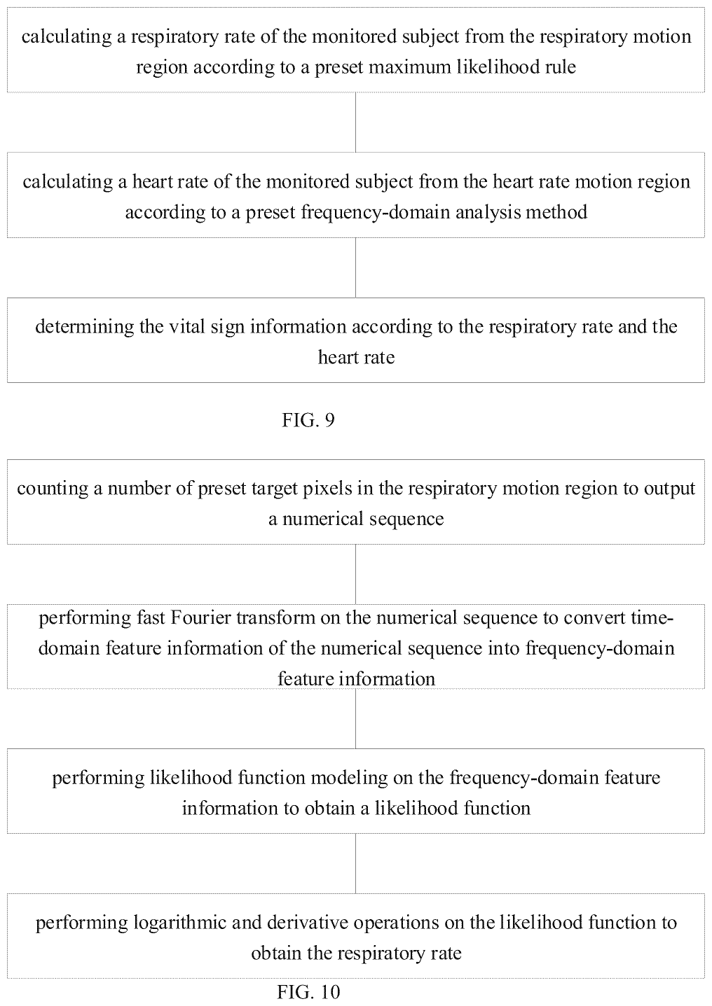

Preferably, referring to FIG. 9, the step of performing vital sign extraction on the target region to obtain vital sign information of the monitored subject comprises:
calculating a respiratory rate of the monitored subject from the respiratory motion region according to a preset maximum likelihood rule;

Specifically, the maximum likelihood rule refers to selecting, among a set of candidate frequencies, the one that makes the observed time-series signal most probable. The respiratory motion region is the area previously determined in which chest-abdominal surfaces or covered surfaces exhibit the most evident respiratory oscillations. The purpose is to obtain a more robust respiratory fundamental frequency under conditions of low SNR and short observation windows;

In implementation, a one-dimensional time-series signal from this region is first detrended and band-pass filtered to cover the typical respiratory frequency range. A fast Fourier transform is then applied to locate the initial spectral peak. Around this peak, a noise hypothesis and likelihood function are constructed, and log-likelihood maximization combined with peak interpolation is performed to refine the optimal frequency. The refined frequency is smoothed over time using a sliding window, and finally converted into breaths per minute with an associated confidence score. This approach is less sensitive to noise, minor body motion, and illumination fluctuations, and provides significantly more stable readings than simple peak-picking methods;
calculating a heart rate of the monitored subject from the heart rate motion region according to a preset frequency-domain analysis method; and determining the vital sign information according to the respiratory rate and the heart rate;

Specifically, the frequency-domain analysis refers to transforming the time-series signal into the frequency domain and identifying the dominant frequency through the power spectrum. The heart rate motion region is typically selected in areas such as the temples or the neck, where pulsations are more evident. The purpose is to accurately isolate subtle periodic components caused by pulse activity;

In implementation, the region time-series signal is constructed using color variation or micro-motion statistics, detrended, and band-pass filtered to cover the typical heart rate band. After windowing, a segmental averaged power spectrum estimation method is applied to obtain a stable spectrum. A dominant peak search and significance verification are then performed on the spectrum, while rejecting spurious peaks associated with respiration and its harmonics. In addition, temporal consistency constraints across adjacent windows and bilateral candidate fusion are employed. The output is expressed as beats per minute with an associated confidence score. This approach maintains high accuracy and continuity even under variations in illumination, skin tone, or minor head movements.

Preferably, referring to FIG. 10, the step of calculating a respiratory rate of the monitored subject from the respiratory motion region according to a preset maximum likelihood rule comprises:
counting a number of preset target pixels in the respiratory motion region to output a numerical sequence;

Specifically, the preset target pixels refer to bright pixels in the difference images whose variation amplitude exceeds a threshold, reflecting local oscillations. The purpose is to compress the subtle respiratory motion in the two-dimensional frames into a one-dimensional time-series, making subsequent frequency estimation more convenient;

In implementation, consecutive frames are first stabilized and denoised to generate difference images. Thresholding and morphological opening/closing are then applied to remove isolated points, after which the number of white pixels is counted frame by frame to form a numerical sequence arranged over time (for example, 39 samples within 20 seconds, equivalent to a 2 Hz sampling rate). This process condenses a large number of pixels into a single indicator, significantly improving both the signal-to-noise ratio and computational efficiency;
performing fast Fourier transform on the numerical sequence to convert time-domain feature information of the numerical sequence into frequency-domain feature information;

Specifically, the numerical sequence is subjected to an FFT (Fast Fourier Transform) to convert it from the time domain to the frequency domain. The transformation formula is:

$$X ( f ) = {\sum }_{n = 0}^{N - 1} x [ n ] {e}^{- 2 π i \frac{{f}_{n}}{N}}$$

where N is the number of samples (in this case, 39), and x[n] represents the value of the numerical sequence at the n-th frame. The purpose of this step is to analyze the frequency components of the numerical sequence in order to identify the dominant periodic variation, namely the respiratory frequency component;
performing likelihood function modeling on the frequency-domain feature information to obtain a likelihood function;

Specifically, it is assumed that the distribution of the respiratory frequency approximates a normal distribution, and the likelihood function is expressed as:

$$L ( f ; x 1 , x 2 , … , xn ) = {\prod }_{i = 1}^{n} \frac{1}{\sqrt{2 πσ}} {e}^{- \frac{{( x i - μ ( f ) )}^{2}}{2 {σ}^{2}}}$$

where (f) is the theoretical mean at frequency f, and a is the standard deviation, x1, x2, . . . , xn represent the observed sample values extracted from the numerical sequence of the respiratory motion region;
performing logarithmic and derivative operations on the likelihood function to obtain the respiratory rate;

Specifically, the above maximum likelihood function is transformed into a log-likelihood function, i.e., ln(L(f;x1, x2, . . . , xn)). The derivative of the log-likelihood function is taken, set equal to zero, and the equation is solved to obtain f. The frequency fff thus determined corresponds to the respiratory information of the monitored subject;

The overall process extracts frequency features from the numerical sequence and, through frequency-domain analysis combined with maximum likelihood estimation, provides an estimate of the subject's respiratory rate. This estimation is based on statistical modeling of the observed data so as to maximize the likelihood of the respiratory frequency;

Preferably, referring to FIG. 11, the step of calculating a heart rate of the monitored subject from the heart rate motion region according to a preset frequency-domain analysis method comprises:
extracting pixel intensity variations of the heart rate motion region to construct a one-dimensional signal sequence for frequency-domain analysis;

Specifically, the heart rate motion region is typically selected in areas where pulsations are prominent, such as the temples or the sides of the neck. The purpose of this step is to convert the subtle temporal fluctuations in brightness or color within the region into an analyzable univariate sequence;

In implementation, the grayscale mean, weighted mean of skin-color channels, color differences, or median intensity of micro-displacements can be calculated frame by frame within the region. Minor smoothing may be applied to suppress random noise, thereby obtaining a one-dimensional sequence arranged in temporal order. To reduce the influence of illumination variations, color combinations more robust to lighting changes or pixel subsets with higher contrast may be prioritized. This conversion compresses the two-dimensional frame information into a time series, significantly improving both the efficiency and the signal-to-noise ratio of subsequent spectral analysis;
performing preprocessing on the one-dimensional signal sequence to obtain a preprocessed signal sequence, wherein the preprocessing comprises eliminating a direct current component, removing a trend term, and performing windowing;

Specifically, the purpose of preprocessing is to make the heart rate-related periodic components more distinguishable in the frequency domain. The procedure comprises first eliminating the DC component by subtracting the mean from the sequence; then removing the trend term, for example by subtracting a low-order polynomial fit or applying a high-pass method to eliminate slow drifts; and subsequently applying windowing, commonly using a Hanning or Hamming window, to reduce leakage effects caused by truncation. If necessary, amplitude normalization and outlier clipping may also be performed. The preprocessed sequence effectively suppresses slow illumination variations and sporadic disturbances, thereby concentrating the true heart rate energy within the target frequency band and making peak identification more reliable;
performing calculation on the preprocessed signal sequence according to a preset segmental averaged power spectrum estimation method to obtain a power spectrum distribution within a preset frequency range;

Specifically, the time series is divided into a plurality of overlapping short segments, each segment is windowed and its power spectrum is computed, and the spectra of the segments are then averaged to obtain a stable power spectrum distribution. The purpose of this step is to improve the robustness of spectrum estimation under short-time windows and low signal-to-noise conditions, thereby reducing the impact of random noise on a single spectral estimate;

During the calculation, only the spectral lines within the preset heart rate frequency range are retained, and, if necessary, spectral smoothing and out-of-band suppression are applied. The amplitude relationship between the dominant peak and neighboring peaks is also recorded to support subsequent significance and confidence evaluation. The final in-band power spectrum is both smoothed and sufficiently resolved, laying the foundation for accurate localization of the dominant peak;
determining a target frequency corresponding to a power peak in the power spectrum distribution, and correcting the target frequency by quadratic interpolation to obtain a corrected target frequency; and converting the corrected target frequency into a heart rate value to obtain the heart rate;

Specifically, the frequency with the maximum amplitude within the in-band power spectrum is identified as the initial value of the target frequency. Quadratic interpolation (parabolic fitting) is then performed using the peak and its neighboring points to refine the frequency from discrete grid points to higher precision. To avoid misjudgment, checks may also be applied to verify that the distance from the respiratory frequency and its harmonics, as well as the peak width and significance, satisfy preset thresholds. After obtaining the corrected target frequency, multiplying it by sixty yields the heart rate in beats per minute, and a confidence score may be provided based on the ratio of the peak to the in-band background. This approach produces heart rate readings that are more stable and accurate under noisy, short-window, or slight-motion conditions, thereby facilitating continuous monitoring and early warning.

Preferably, after calculating the respiratory rate of the monitored subject from the respiratory motion region according to the preset maximum likelihood rule to obtain the respiratory rate, the method further comprises:
determining a real-time sleep stage of the monitored subject according to the respiratory information of the monitored subject and a preset respiratory rate range associated with the sleep stages of the monitored subject, wherein the sleep stages at least comprise a deep sleep stage and a light sleep stage;
judging, based on the respiratory rate of the monitored subject, whether the subject is currently in a deep sleep state or a light sleep state;

Specifically, by monitoring the respiratory rate, it is possible to assist in determining whether the monitored subject is in a deep sleep state (slower respiratory rate) or a light sleep state (faster respiratory rate). For example, assuming that the normal respiratory rate range of the monitored subject during deep sleep is 15-20 breaths per minute, and the respiratory rate range during light sleep is 20-25 breaths per minute, when the monitored subject is detected to have a respiratory rate of 17 breaths per minute, it can be determined, based on the preset ranges, that the monitored subject is currently in a deep sleep state;
determining, according to time information, whether the monitored subject is in a nap stage or a nighttime sleep stage;

Specifically, time information is used to determine the current sleep stage so as to support further analysis. For example, during a first preset time period in the daytime, the subject is recognized as being in a nap stage, while during a second preset time period at night, the subject is recognized as being in a nighttime sleep stage. The first preset time period and the second preset time period can be set and adaptively adjusted according to actual needs. This distinction is of great significance for analyzing the overall sleep patterns and behaviors of the monitored subject, since nap sleep and nighttime sleep usually differ in duration and sleep depth;
if the monitored subject is in the nap stage and the real-time sleep stage is the deep sleep stage, acquiring a deep sleep duration;

Specifically, when it is detected that the monitored subject is in the nap stage and determined to have entered the deep sleep stage, the duration of deep sleep is recorded. This process is carried out automatically and updated in real time to ensure the accuracy of the monitoring data. The deep sleep duration is an important indicator for evaluating the quality of a nap and has a direct impact on the physical recovery and growth of the monitored subject. Therefore, accurately capturing and recording this duration helps parents or caregivers to understand the sleep condition of the monitored subject and take timely measures for improvement;
comparing the deep sleep duration with a preset duration threshold to output a comparison result;

Specifically, the actual deep sleep duration of the monitored subject is compared with a preset ideal duration threshold, which is set according to factors such as the age and health condition of the monitored subject. The comparison result indicates whether the current deep sleep meets the required standard. If the deep sleep duration does not reach the preset threshold, parents or caregivers may be prompted to take corresponding measures, such as adjusting schedules or improving the sleep environment, so as to help the monitored subject obtain higher-quality sleep;
if the monitored subject is in the nighttime sleep stage, acquiring the deep sleep duration and the light sleep duration;

Specifically, for the nighttime sleep stage, not only the deep sleep duration is monitored, but also the light sleep duration is recorded. This is because nighttime sleep generally constitutes the main sleep cycle of the monitored subject, involving alternations between deep and light sleep. By simultaneously acquiring both types of sleep duration, a more comprehensive evaluation of nighttime sleep quality can be achieved. Such monitoring helps identify potential sleep problems, such as insufficient deep sleep or excessive light sleep, which may affect the growth and development of the monitored subject;
determining a sleep cycle ratio according to the deep sleep duration and the light sleep duration;
outputting reminder information according to the comparison result and the sleep cycle ratio;

Specifically, the sleep cycle ratio of the monitored subject is analyzed, that is, the relative ratio between the deep sleep duration and the light sleep duration. A normal sleep cycle usually involves alternations of deep and light sleep in certain proportions, which are crucial for physical recovery and consolidation of brain functions. By calculating this ratio, abnormal sleep patterns can be identified, such as insufficient deep sleep or excessive light sleep, and corresponding feedback can be provided to help parents improve the sleep habits of the monitored subject.

Preferably, the duration threshold is set according to the age and health condition of the monitored subject, and outputting reminder information according to the comparison result and the sleep cycle ratio comprises:
if the deep sleep duration is less than the duration threshold and/or the sleep cycle ratio is abnormal, outputting the reminder information.

Specifically, based on the analysis results of the previous steps, particularly the comparison result between the deep sleep duration and the ideal threshold as well as the sleep cycle ratio, reminder information is provided to parents or caregivers. If the monitored subject is found to have unsatisfactory sleep quality, such as insufficient deep sleep or excessive light sleep, suggestions or prompts may be delivered in the form of notifications or alarms. Such reminders help parents to timely understand the sleep condition of the monitored subject and take necessary intervention measures to ensure that the monitored subject obtains sufficient and high-quality sleep.

### Embodiment 2

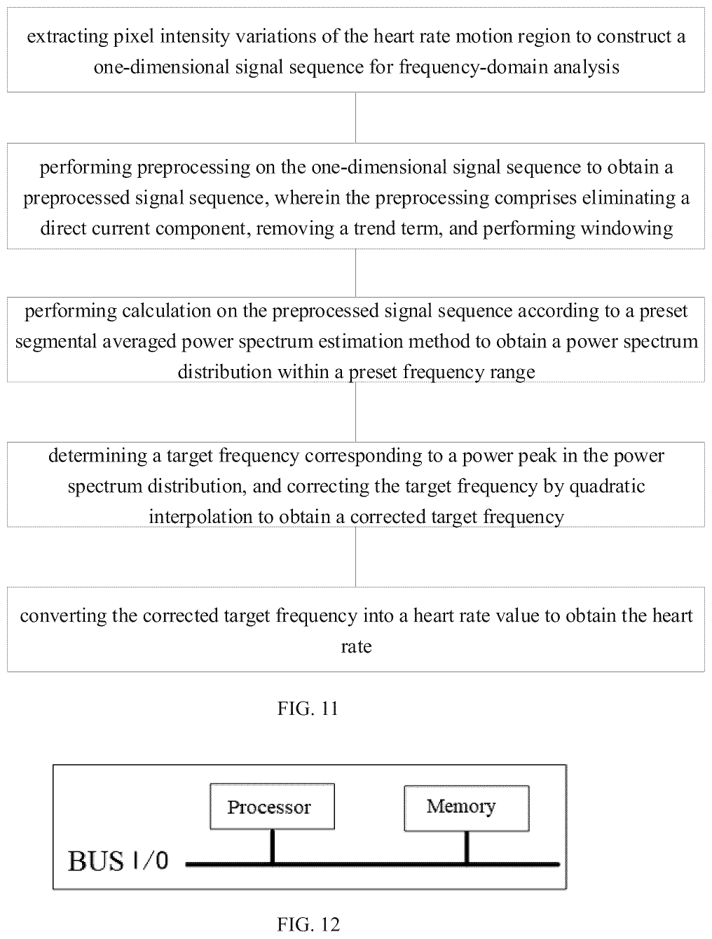

In addition, the non-invasive vital sign monitoring method of Embodiment 1 of the present invention, as described in connection with FIG. 1, may be implemented by an electronic device. FIG. 12 illustrates a schematic diagram of the hardware structure of an electronic device provided in Embodiment 2 of the present invention.

The electronic device may comprise a processor and a memory storing computer program instructions.

Specifically, the processor may include a Central Processing Unit (CPU), or an Application Specific Integrated Circuit (ASIC), or one or more integrated circuits configured to implement one or more embodiments of the present invention.

The memory may comprise a mass storage device for data or instructions. By way of example and not limitation, the memory may comprise a Hard Disk Drive (HDD), a floppy disk drive, a flash memory, an optical disk, a magneto-optical disk, a magnetic tape, or a Universal Serial Bus (USB) drive, or a combination of two or more thereof. Where appropriate, the memory may comprise removable or non-removable (fixed) media. Where appropriate, the memory may be internal or external to a data processing device. In particular embodiments, the memory is non-volatile solid-state memory. In particular embodiments, the memory comprises Read Only Memory (ROM). Where appropriate, the ROM may be a mask-programmed ROM, a Programmable ROM (PROM), an Erasable PROM (EPROM), an Electrically Erasable PROM (EEPROM), an Electrically Alterable ROM (EAROM), flash memory, or a combination of two or more thereof.

The processor reads and executes the computer program instructions stored in the memory so as to implement any of the foregoing embodiments of the non-invasive vital sign monitoring method.

In one example, the electronic device may further comprise a communication interface and a bus. As shown in FIG. 11, the processor, the memory, and the communication interface are connected through the bus and complete mutual communication.

The communication interface is mainly used to realize communication among the modules, devices, units, and/or equipment in the embodiments of the present invention.

The bus includes hardware, software, or both, and couples the components of the device to each other. By way of example and not limitation, the bus may comprise an Accelerated Graphics Port (AGP) or other graphics bus, an Enhanced Industry Standard Architecture (EISA) bus, a Front Side Bus (FSB), a HyperTransport (HT) interconnect, an Industry Standard Architecture (ISA) bus, an InfiniBand interconnect, a Low Pin Count (LPC) bus, a memory bus, a MicroChannel Architecture (MCA) bus, a Peripheral Component Interconnect (PCI) bus, a PCI-Express (PCI-X) bus, a Serial Advanced Technology Attachment (SATA) bus, a Video Electronics Standards Association (VLB) local bus, or any other suitable bus, or a combination of two or more thereof. Where appropriate, the bus may comprise one or more buses. Although the embodiments of the present invention describe and illustrate specific buses, the present invention contemplates any suitable bus or interconnect.

### Embodiment 3

In addition, in combination with the non-invasive vital sign monitoring method in Embodiment 1, Embodiment 3 of the present invention further provides computer program product comprising program instruction that are stored on a computer-readable medium and that, when executed by a processor, cause an electronic device to implement any of the foregoing embodiments of the non-invasive vital sign monitoring method.

In summary, the embodiments of the present invention provide a non-invasive vital sign monitoring method, an electronic device, and a computer program product.

It should be understood that the present invention is not limited to the specific configurations and processes described above and shown in the drawings. For the sake of brevity, detailed descriptions of well-known methods have been omitted. In the foregoing embodiments, several specific steps are described and illustrated as examples. However, the method process of the present invention is not limited to the specific steps described and illustrated. Those skilled in the art may, upon understanding the spirit of the invention, make various changes, modifications, and additions, or alter the order of the steps.

The functional blocks shown in the structural diagrams above may be implemented in hardware, software, firmware, or a combination thereof. When implemented in hardware, they may comprise, for example, electronic circuits, application-specific integrated circuits (ASICs), appropriate firmware, plug-ins, function cards, and the like. When implemented in software, the elements of the present invention are programs or code segments configured to perform the required tasks. The program or code segment may be stored in a machine-readable medium or transmitted over a transmission medium or communication link via a data signal carried on a carrier wave. Examples of “machine-readable media” include any medium capable of storing or transmitting information, such as electronic circuits, semiconductor memory devices, ROM, flash memory, erasable ROM (EROM), floppy disks, CD-ROMs, optical disks, hard disks, optical fiber media, radio-frequency (RF) links, and the like. The code segments may be downloaded via a computer network such as the Internet or an intranet.

The user information (including but not limited to user device information and personal information) and data (including but not limited to data for analysis, storage, and display) involved in this application are all information and data authorized by the user or duly authorized by all parties, and the collection, use, and processing of such data must comply with relevant local laws, regulations, and standards. Appropriate operation interfaces are provided to allow users to choose whether to authorize or refuse.

It should also be noted that the exemplary embodiments of the present invention describe certain methods or systems based on a series of steps or devices. However, the present invention is not limited to the order of the steps. That is, the steps may be performed in the order described in the embodiments, in a different order, or certain steps may be executed simultaneously.

The foregoing description is merely specific embodiments of the present invention. Those skilled in the art will clearly understand that, for the sake of convenience and brevity, the specific working processes of the systems, modules, and units described above may refer to the corresponding processes in the foregoing method embodiments and will not be repeated here. It should be understood that the scope of protection of the present invention is not limited thereto. Any equivalent modifications or substitutions conceived by those skilled in the art within the technical scope disclosed by the present invention shall fall within the scope of protection of the present invention.

What is claimed is:

1. A non-invasive vital sign monitoring method, wherein the method comprises:
acquiring a real-time infrared video image in a sleep monitoring scenario;
performing sleep state monitoring on a monitored subject in the real-time infrared video image to obtain a first target image indicating that the monitored subject has entered a sleep state;
performing motion magnification processing on the first target image according to preset vital sign information to obtain a second target image;
performing target region extraction on the second target image to obtain a target region associated with the preset vital sign information; and,
performing vital sign extraction on the target region to obtain vital sign information of the monitored subject.

2. The non-invasive vital sign monitoring method according to claim 1, wherein performing sleep state monitoring on a monitored subject in the real-time infrared video image to obtain a first target image indicating that the monitored subject has entered a sleep state comprises:
inputting the real-time infrared video image into a pre-trained object detection model to acquire a head position of the monitored subject;
performing object tracking on the head position of the monitored subject to output head tracking box information;
determining, based on the head tracking box information, whether the monitored subject has entered a sleep state; and,
if it is determined that the monitored subject has entered the sleep state, taking the real-time infrared video image in which the monitored subject has entered the sleep state as the first target image.

3. The non-invasive vital sign monitoring method according to claim 2, wherein determining, based on the head tracking box information, whether the monitored subject has entered a sleep state comprises:
acquiring center position coordinates of head tracking boxes based on the head tracking box information;
performing mean value calculation on the center position coordinates to obtain average position information;
determining a head movement state of the monitored subject according to the center position coordinates and the average position information;
acquiring a number of consecutive frames of the real-time infrared video image in which a head of the monitored subject is in a moving state according to the head movement state;
if the number of consecutive frames of the real-time infrared video image in which the head of the monitored subject is in a moving state is greater than a preset frame threshold, identifying that the monitored subject has not entered the sleep state; and,
if the number of consecutive frames of the real-time infrared video image in which the head of the monitored subject is in a moving state is less than or equal to the preset frame threshold, identifying that the monitored subject has entered the sleep state.

4. The non-invasive vital sign monitoring method according to claim 1, wherein performing motion magnification processing on the first target image according to preset vital sign information to obtain a second target image comprises:
performing grayscale conversion on the first target image to acquire a grayscale image;
performing filtering on the grayscale image to output a filtered image;
performing Fourier transform on the filtered image to output an initial frequency-domain image;
enhancing a target frequency in the initial frequency-domain image associated with the preset vital sign information to output an enhanced target frequency-domain image, wherein the preset vital sign information comprises respiratory motion and heart rate motion; and
performing inverse Fourier transform on the target frequency-domain image to output the second target image.

5. The non-invasive vital sign monitoring method according to claim 4, wherein enhancing a target frequency in the initial frequency-domain image associated with the preset vital sign information to output an enhanced target frequency-domain image comprises:
enhancing, according to a first preset frequency range corresponding to respiratory motion, the target frequency in the initial frequency-domain image associated with the respiratory motion to obtain a first frequency-domain image in which the respiratory motion is enhanced;
enhancing, according to a second preset frequency range corresponding to heart rate motion, the target frequency in the initial frequency-domain image associated with the heart rate motion to obtain a second frequency-domain image in which the heart rate motion is enhanced; and,
determining the target frequency-domain image based on the first frequency-domain image and the second frequency-domain image.

6. The non-invasive vital sign monitoring method according to claim 1, wherein performing target region extraction on the second target image to obtain a target region associated with the preset vital sign information comprises:
performing respiratory motion region extraction on the second target image to obtain a respiratory motion region;
performing heart rate motion region extraction on the second target image to obtain a heart rate motion region; and,
determining the target region based on the respiratory motion region and the heart rate motion region.

7. The non-invasive vital sign monitoring method according to claim 6, wherein the performing respiratory motion region extraction on the second target image to obtain a respiratory motion region comprises:
acquiring a preset number of consecutive frames of the second target image and classifying the second target image into a plurality of image sets;
performing pixel-wise averaging on the image sets to output average images corresponding to each image set;
performing difference calculation on the average images to obtain difference images; and
determining the respiratory motion region based on pixel differences in the difference images and a preset pixel threshold.

8. The non-invasive vital sign monitoring method according to claim 7, wherein determining the respiratory motion region based on pixel differences in the difference image and a preset pixel threshold comprises:
taking, according to the pixel differences and the pixel threshold, pixels having pixel differences greater than the pixel threshold as target pixels, and acquiring position information of the target pixels in each difference image;
determining contour regions based on the position information of the target pixels;
comparing areas of the contour regions in the difference images and taking a largest contour region as a target contour region; and,
performing minimum bounding rectangle extraction on the target contour region to obtain the respiratory motion region.

9. The non-invasive vital sign monitoring method according to claim 6, wherein the performing heart rate motion region extraction on the second target image to obtain the heart rate motion region comprises:
acquiring center position coordinates and size information of head tracking boxes based on the head tracking box information;
calculating, according to the center position coordinates and the size information, to obtain neck region position information and temple region position information;
performing quality evaluation on neck candidate regions and temple candidate regions according to the neck region position information and the temple region position information to obtain a quality evaluation result; and,
screening, based on the quality evaluation result, the neck candidate region or the temple candidate region as the heart rate motion region.

10. The non-invasive vital sign monitoring method according to claim 9, wherein calculating, according to the center position coordinates and the size information, to obtain neck region position information and temple region position information comprises:
acquiring a width and a height of the head tracking box according to the size information;
determining a first longitudinal boundary of the neck region based on the center position coordinate and the height in combination with a first preset ratio coefficient and a second preset ratio coefficient;
determining a first transverse boundary of the neck region based on the center position coordinate and the width in combination with a third preset ratio coefficient; and
determining the neck region position information according to the first transverse boundary and the first longitudinal boundary.

11. The non-invasive vital sign monitoring method according to claim 10, wherein calculating, according to the center position coordinates and the size information, to obtain neck region position information and temple region position information comprises:
determining, based on the center position coordinate and the height in combination with a fourth preset ratio coefficient, a second transverse boundary and a second longitudinal boundary of the temple region at an outer side position of an upper half portion of the head tracking box; and
determining the temple region position information according to the second transverse boundary and the second longitudinal boundary.

12. The non-invasive vital sign monitoring method according to claim 9, wherein the performing quality evaluation on neck candidate regions and temple candidate regions according to the neck region position information and the temple region position information to obtain a quality evaluation result comprises:
extracting pixel data of consecutive frames from the neck candidate regions and the temple candidate regions according to the neck region position information and the temple region position information, and constructing region time-series data for evaluation;
performing imaging quality evaluation on the region time-series data to obtain a first quality metric;
performing energy analysis on the region time-series data according to a preset heart rate-related frequency band to obtain a second quality metric, the energy analysis being configured to characterize the strength relationship between a dominant frequency component and in-band background components;
performing stability evaluation on the region time-series data using a preset time segmentation manner to obtain a third quality metric, the stability evaluation being configured to characterize the consistency of heart rate candidate frequencies across different time segments;
performing artifact detection on the region time-series data to obtain a fourth quality metric, the artifact detection comprising correlation detection with a respiratory rate and harmonics thereof and correlation detection with global body motion; and,
performing weighted calculation based on the first quality metric, the second quality metric, the third quality metric, and the fourth quality metric to obtain the quality evaluation result.

13. The non-invasive vital sign monitoring method according to claim 6, wherein performing vital sign extraction on the target region to obtain the vital sign information of the monitored subject comprises:
calculating a respiratory rate of the monitored subject from the respiratory motion region according to a preset maximum likelihood rule;
calculating a heart rate of the monitored subject from the heart rate motion region according to a preset frequency-domain analysis method; and
determining the vital sign information according to the respiratory rate and the heart rate.

14. The non-invasive vital sign monitoring method according to claim 13, wherein calculating a respiratory rate of the monitored subject from the respiratory motion region according to a preset maximum likelihood rule comprises:
counting a number of preset target pixels in the respiratory motion region to output a numerical sequence;
performing fast Fourier transform on the numerical sequence to convert time-domain feature information of the numerical sequence into frequency-domain feature information;
performing likelihood function modeling on the frequency-domain feature information to obtain a likelihood function; and,
performing logarithmic and derivative operations on the likelihood function to obtain the respiratory rate.

15. The non-invasive vital sign monitoring method according to claim 13, wherein calculating a heart rate of the monitored subject from the heart rate motion region according to a preset frequency-domain analysis method comprises:
extracting pixel intensity variations of the heart rate motion region to construct a one-dimensional signal sequence for frequency-domain analysis;
performing preprocessing on the one-dimensional signal sequence to obtain a preprocessed signal sequence, wherein the preprocessing comprises eliminating a direct current component, removing a trend term, and performing windowing;
performing calculation on the preprocessed signal sequence according to a preset segmental averaged power spectrum estimation method to obtain a power spectrum distribution within a preset frequency range;
determining a target frequency corresponding to a power peak in the power spectrum distribution, and correcting the target frequency by quadratic interpolation to obtain a corrected target frequency; and
converting the corrected target frequency into a heart rate value to obtain the heart rate.

16. The non-invasive vital sign monitoring method according to claim 13, wherein after calculating a respiratory rate of the monitored subject from the respiratory motion region according to the preset maximum likelihood rule to obtain the respiratory rate, the method further comprises:
determining a real-time sleep stage of the monitored subject according to the respiratory rate of the monitored subject and a preset respiratory rate range associated with human sleep stages, wherein the sleep stages comprise a deep sleep stage and a light sleep stage;
determining, according to time information, whether the monitored subject is in a nap stage or a nighttime sleep stage;
if the monitored subject is in the nap stage and the real-time sleep stage is the deep sleep stage, acquiring a deep sleep duration;
comparing the deep sleep duration with a preset duration threshold to output a comparison result;
if the monitored subject is in the nighttime sleep stage, acquiring the deep sleep duration and a light sleep duration;
determining a sleep cycle ratio according to the deep sleep duration and the light sleep duration; and,
outputting reminder information according to the comparison result and the sleep cycle ratio.

17. The non-invasive vital sign monitoring method according to claim 16, wherein the duration threshold is set according to the age and health condition of the monitored subject, and outputting reminder information according to the comparison result and the sleep cycle ratio comprises:
if the deep sleep duration is less than the duration threshold and/or the sleep cycle ratio is abnormal, outputting the reminder information.

18. The non-invasive vital sign monitoring method according to claim 1, wherein acquiring a real-time infrared video image in a sleep monitoring scenario comprises:
acquiring real-time infrared video data including the monitored subject in the sleep monitoring scenario; and,
decomposing the real-time infrared video data to obtain a plurality of frames of the real-time infrared video image.

19. An electronic device, wherein the electronic device comprises:
at least one processor;
at least one memory; and,
computer program instructions stored in the memory, which, when executed by the processor, implement the method comprising:
acquiring a real-time infrared video image in a sleep monitoring scenario;
performing sleep state monitoring on a monitored subject in the real-time infrared video image to obtain a first target image indicating that the monitored subject has entered a sleep state;
performing motion magnification processing on the first target image according to preset vital sign information to obtain a second target image;
performing target region extraction on the second target image to obtain a target region associated with the preset vital sign information; and,
performing vital sign extraction on the target region to obtain vital sign information of the monitored subject.

20. A computer program product comprising program instruction that are stored on a computer-readable medium and that, when executed by a processor, cause an electronic device to implement the method comprising:
acquiring a real-time infrared video image in a sleep monitoring scenario;
performing sleep state monitoring on a monitored subject in the real-time infrared video image to obtain a first target image indicating that the monitored subject has entered a sleep state;
performing motion magnification processing on the first target image according to preset vital sign information to obtain a second target image;
performing target region extraction on the second target image to obtain a target region associated with the preset vital sign information; and,
performing vital sign extraction on the target region to obtain vital sign information of the monitored subject.
